(19) United States
(12) Patent Application Publication
(10) Pub. No.: US 2019/0270971 A1
(43) Pub. Date: Sep. 5, 2019

(54) INCREASING PRODUCTIVITY OF MICROBIAL HOST CELLS THAT FUNCTIONALLY EXPRESS P450 ENZYMES

(71) Applicants: Jason DONALD, Cambridge, MA (US); Ajikumar Parayil KUMARAN, Cambridge, MA (US); Christine Nicole S. SANTOS, Cambridge, MA (US)

(72) Inventors: Jason DONALD, Cambridge, MA (US); Ajikumar Parayil KUMARAN, Cambridge, MA (US); Christine Nicole S. SANTOS, Cambridge, MA (US)

(21) Appl. No.: 16/288,342

(22) Filed: Feb. 28, 2019

Related U.S. Application Data
(60) Provisional application No. 62/637,125, filed on Mar. 1, 2018.

(51) Int. Cl. C12N 9/02; C07K 14/705; C12P 19/56

(52) U.S. Cl. CPC C12N 9/0071; C12Y 114/00; C07K 2319/03; C12P 19/56; C07K 14/70521

(57) ABSTRACT

The present technology relates to the production of chemical species in bacterial or yeast host cells. Particularly, the present technology provides for the production of chemical species in microbial host cells that functionally express engineered P450 enzymes.

### FIELD

The present technology relates to production of chemical species through oxidative chemistry in microbial host cells. Particularly, the present technology provides P450 enzymes engineered for functional expression in microbial host cells.

### BACKGROUND

Microbes (e.g., bacteria and yeast) are widely used for production of chemicals by the recombinant expression of biosynthetic pathways, which can involve overexpression of several native and/or foreign genes.

It is an object of the present technology to improve productivity of P450 enzymes in microbial platforms to thereby expand the utility of these platforms for P450 chemistry.

### SUMMARY

In various aspects, the present technology provides P450 enzymes engineered for functional expression in microorganisms and polynucleotides encoding the same. The present technology further provides microbial host cells expressing the engineered P450 enzymes, and methods for producing chemical species through recombinant expression of biosynthetic pathways involving P450 enzymes.

The engineered P450 enzymes described herein have a deletion of all or part of the wild type P450 N-terminal transmembrane region, and the addition of a transmembrane domain derived from a microbial (e.g., bacteria or yeast) inner membrane, cytoplasmic C-terminus protein. It is believed that the transmembrane domain acts to anchor the P450 in a microbial (e.g., bacteria or yeast) membrane, such, e.g., the inner membrane. In some embodiments, the transmembrane domain is a single-pass transmembrane domain. In some embodiments, the transmembrane domain is a multi-pass transmembrane domain.

In some aspects, the present technology provides methods for the production of chemical species by expressing in microbial cells one or more biosynthetic pathways including at least one membrane-anchored P450 (CYP) enzyme, and culturing the microbial cells to produce the chemical species. At least one membrane-anchored P450 enzyme contains a transmembrane domain derived from a microbial (e.g., bacteria or yeast) inner membrane, cytoplasmic C-terminus protein, which may be a cytoplasmic C-terminus protein from the host genus or species. Previous methods for expressing P450 proteins in microbes can result in a substantial stress response, which limits productivity of the microbial host cell. In some embodiments, microbial cells expressing the engineered P450 enzymes described herein do not exhibit a substantially stressed phenotype in some embodiments, thereby improving pathway productivity.

The present technology in various aspects is applicable to various P450 enzymes, including plant-derived P450 enzymes, which can be further engineered for productivity in a microbial host cell system. These engineered P450 enzymes can be used in the production of a variety of chemical species through recombinant pathway expression, including but not limited to production of terpenoid compounds. Terpenoids represent a diverse class of molecules that provide beneficial health and nutritional attributes, as well as numerous other commercial applications. For example, terpenoids find use in the food and beverage industries as well as the perfume, cosmetic, pest control, and health care industries.

In various embodiments, the microbial cell is used for the production of chemicals by the recombinant expression of biosynthetic pathways, which can involve overexpression of several native and/or foreign genes. Often, expression of several foreign genes in the microbial cell and/or overexpression of native the microbial genes can induce a substantial stress response, which limits productivity. Conventional expression of P450 enzymes in the microbial cell, together with cytochrome P450 reductase (CPR) partners to regenerate the cofactor, can substantially add to this stress response, as exhibited for example by overexpression of IbpA, a protein that is overexpressed in the microbial cell under conditions of high protein aggregation and stress. It is critical that the P450 enzyme expression induce as little cell stress as possible to avoid limits on pathway productivity. Accordingly, the present technology helps minimize cellular stress in a microbial host cell to increase productivity of the microbial host cell for production of chemical species.

Other aspects and embodiments of the present technology will be apparent from the following detailed disclosure.

### DESCRIPTION OF THE FIGURES

FIG. 1 illustrates exemplary N-terminal anchors for expressing P450 proteins in bacteria or yeast, including the previous designs based on truncation of the P450 transmembrane helix with the addition of an 8-amino acid peptide (8RP), and the use of single-pass and multi-pass transmembrane helices from bacterial or yeast proteins as described herein.

FIG. 2 is a diagram taken from Schramm, et al., Structure, 20(5): 924-35 (2012) that shows the use of E(z) potential to estimate the effect of different N-terminal sequences on anchoring P450's to the membrane, including modeling the three dimensional depth and tilt of the sequence in the membrane.

FIG. 3 is diagram showing that E(z) potential predicts that a wild type SrKO (a Stevia rebaudiana P450 enzyme)N-terminus and a modified SrKO N-terminus that comprises a truncated wild type N-terminus that is fused to the modified N-terminus bovine 17α membrane anchor, will not span the membrane.

FIG. 4 is diagram showing that E(z) potential predicts that a modified wild type SrKO N-terminus that comprises a truncated wild type N-terminus that is fused to an N-terminal anchor of E. coli yhhM membrane anchor will span the membrane.

### DETAILED DESCRIPTION

The present technology provides for improved functional expression of P450 enzymes in microbial host cells (e.g., bacteria and yeast). In some embodiments, the microbial host cells do not naturally possess P450 enzymes, such as E. coli. While some microbial platforms are widely used for production of a wide variety of chemicals, they are generally considered insufficient when P450 chemistry is required.

P450 expression in microbial host cells is typically obtained by co-expression of the P450 with a CPR. The P450 enzyme contains a truncation of at least part of the native P450 transmembrane region, which is replaced with the 8 amino acid tag MALLLAVF (which is derived from bovine P450 17α) at the N-terminus. The present technology demonstrates that this tag is far from optimal, and results in a substantial cell stress response.

The present technology provides P450 enzymes engineered for functional expression in microbial platforms. In some embodiments, the P450 enzymes comprise an N-terminal membrane anchor sequence derived from a native microbial inner membrane protein having a cytoplasmic C-terminus. In some embodiments, the N-terminal membrane anchor sequence replaces some or the entire native P450 N-terminal transmembrane region, where present in the wild-type enzyme. In some embodiments, expression of the engineered P450 enzymes in the microbial host cell induces less cell stress than previous attempts to functionally express P450 enzymes. The present technology allows for increases in biosynthetic productivity in microbial host platforms, due in-part, to substantial improvements in P450 efficiency and to minimizing the host cell stress response.

In some embodiments, the P450 enzyme of the present technology is derived from any source, including plants, animals, or microbes. In some embodiments, the P450 enzyme is selected from a CYP70, CYP71, CYP73, CYP76 (e.g., CYP76F), CYP82 or CYP92 family P450. In some embodiments, the P450 is an enzyme disclosed in U.S. Pat. No. 8,722,363, which is hereby incorporated by reference. In some embodiments, the P450 is a plant P450 enzyme. Plant cytochrome P450s are involved in a wide range of biosynthetic reactions, leading to various fatty acid conjugates, plant hormones, defensive compounds, or medically and commercially important compounds, including terpenoids. Terpenoids represent the largest class of characterized natural plant compounds and are often substrates for plant P450 enzymes. In some embodiments, the P450 is derived from a species selected from Zingibersp., Barnadesia sp., Hyoscyamus sp., Latuca sp., Nicotiana sp., Citrus sp., Siraitia sp., Artemesia sp., Arabidopsis sp, Stevia sp., Bacillus sp., Pleurotus sp., Cichorium sp., Helianthus sp., and Physcomitrella sp., Taxus sp., Rosa sp., Cymbopogon sp., Humulus sp., Pogostemon sp., and Cannabis sp. Wild-type P450 enzyme sequences are known and publically available, and/or can be obtained by genetic analysis of select plants based on well-known P450 motifs. See, for example, Saxena A. et al., Identification of cytochrome P450 heme motif in plants proteome, Plant Omics (2013); Chapple C., Molecular-genetic analysis of plant cytochrome p450-dependent monooxygenases, Annual Review of Plant Physiology and Plant Molecular Biology Vol. 49:311-343 (1998).

Table 1 provides a list of exemplary P450 enzymes that may be engineered in accordance with the present technology:

TABLE 1

| Species | Name | Native Substrate | Native Reaction Product |
|---|---|---|---|
| Zingiber | zzHO | α-humulene | 8-hydroxy-α-humulene |
| zerumbet | | | |
| Barnadesia | BsGAO | germacrene A | germacra-1(10),4,11(13)- |
| spinosa | | | trien-12-ol |
| Hyoscyamus | HmPO | premnaspirodiene | solavetivol |
| muticus | | | |
| Latuca spicata | LsGAO | germacrene A | germacra-1(10),4,11(13)- |
| | | | trien-12-ol |
| Nicotiana | NtEAO | 5-epi- | capsidiol |
| tabacum | | aristolochene | |
| Citrus × paradisi | CpVO | valencene | nootkatol |
| Artemesia annua | AaAO | amorphadiene | artemisinic |
| | | | acid |
| Arabidopsis | AtKO | kaurene | kaurenoic |
| thaliana | | | acid |
| Stevia | SrKO | kaurene | kaurenoic |
| rebaudiana | | | acid |
| Physcomitrella | PpKO | kaurene | kaurenoic |
| patens | | | acid |
| Bacillus | BmVO | fatty acids | hydroxylated FAs |
| megaterium | | | |
| Pleurotus | PsVO | valencene | nootkatone |
| sapidus | | | |
| Pleurotus | PoLO | unknown | unknown |
| ostreatus | | | |
| Cichorium | CiVO | valencene | nootkatone |
| intybus | | | |
| Helianthus | HaGAO | germacrene A | germacrene A |
| annuus | | | acid |

Thus, the engineered P450 enzyme may be based on wild-type sequences of ZzHO (SEQ ID NO: 1), BsGAO (SEQ ID NO: 2), HmPO (SEQ ID NO: 3), LsGAO (SEQ ID NO: 4), NtEAO (SEQ ID NO: 5), CpVO (SEQ ID NO: 6), AaAO (SEQ ID NO: 7), AtKO (SEQ ID NO: 8), SrKO (SEQ ID NO: 9), PpKO (SEQ ID NO:10), BmVO (SEQ ID NO: 11), PsVO (SEQ ID NO:12), PoLO (SEQ ID NO: 13), CiVO (SEQ ID NO: 14), or HaGAO (SEQ ID NO: 15). In some embodiments, the engineered P450 enzyme are based on P450 enzymes disclosed in U.S. Application No. 62/635,751, the contents of which are incorporated by reference in its entirety, which provide for the production of mogrosides.

Additional P450 enzymes that can be engineered in accordance with the present technology include limonene-6-hydroxylase (AAQ18706.1, AAD44150.1), (−)-limonene-3-hydroxylase (EF426464, AY622319), kaurenoic acid 13-hydroxylase (EU722415.1), carotenoid cleavage dioxygenase (ABY60886.1, BAJ05401.1), beta-carotene hydroxylase (AAA64983.1), amorpha-4,11-diene monoxygenase (DQ315671), taxadiene 5-alpha hydroxylase (AY289209.2), 5-alpha-taxadienol-10-beta-hydroxylase (AF318211.1), taxoid 10-beta hydroxylase (AY563635.1), taxane 13-alpha-hydroxylase (AY056019.1), taxane 14b-hydroxylase (AY188177.1), taxoid 7-beta-hydroxylase (AY307951.1). The amino acid and encoding nucleotide sequences of these enzymes are hereby incorporated by reference. Derivatives of these P450s may be constructed in accordance with this disclosure.

The particular P450 enzyme scaffold can be selected based on the desired substrate specificity, which may be its natural substrate, or a non-natural substrate similar to the natural substrate, or otherwise determined experimentally. P450's can have varying substrate specificities, and thus can be engineered for chemistry on non-natural substrates. See, for example, Wu et al., Expansion of substrate specificity of cytochrome P450 2A6 by random and site-directed mutagenesis, J. Biol. Chem. 280(49): 41090-100 (2005). Exemplary substrates for P450 chemistry include various secondary metabolites such as, without limitation, terpenoids, alkaloids, cannabinoids, steroids, saponins, glycosides, stilbenoids, polyphenols, antibiotics, polyketides, fatty acids, and non-ribosomal peptides. Exemplary products that may be produced through P450 chemistry include, without limitation, lutein, tocopherol, abietic acid, mogroside, forskolin, amyrin, lupeol, butyrospermol, quillic acid, triterpenoid saponins, oleanic acid, betulinic acid, boswellic acid, gymnemic acid, banaba/corosolic acid, cissus keto-steroid, curcurbitane triterpenoid, santalol, marrubiin, montbretin A, tropolone, sclareol, pseudolaric acid, grindelic acid, kauralexin, viteagnusin, diterpenoid epoxide triptolide, quinone triterpene celastrol, gibberellic acid, pseudolaric acid, carveol, carvone, nootkatol, nootkatone, piperitone, steviol, mogrosides, perillaldehyde, tagetone, verbenone, menthol, thymol, 3-oxo-alpha-lonone, zeanthin, artemisinin, taxol, gingkolide, gossypol, pseudoterosin, crotophorbolone, englerin, psiguadial, stemodinone, maritimol, cyclopamine, veratramine, aplyviolene, macfarlandin E, betulinic acid, oleanolic acid, ursoloic acid, dolichol, lupeol, euphol, cassaic acid, erthroxydiol, trisporic acid, podocarpic acid, retene, dehydroleucodine, phorbol, cafestol, kahweol, tetrahydrocannabinol, androstenol, tanshinone IIA or IIB or VI, cryptotanshinone, 15,16-dihydrotanshinone, trijuganone A or B, dihydrotanshinone I, miltirone, ferruginol, hydrotanshinone IIA, and 1,2-dihydrocrytotanshinone.

Exemplary terpenoid products that may be produced in accordance with the present technology are described in U.S. Pat. No. 8,927,241, which is hereby incorporated by reference, and include: alpha-sinensal, beta-Thujone, Camphor, Carveol, Carvone, Cineole, Citral, Citronellal, Cubebol, Geraniol, Limonene, Menthol, Menthone, Mogrosides, Nootkatone, Nootkatol, Patchouli, Piperitone, Sabinene, Steviol, Steviol glycoside, Taxadiene, and Thymol.

In various embodiments, the engineered P450 enzyme comprises an amino acid sequence that has at least about 30% sequence identity, at least about 40% sequence identity, or at least about 50% sequence identity to any one of SEQ ID NOS: 1-15, or other P450 enzyme described herein. While the P450 need not display high sequence identity to these exemplary P450 enzymes in some embodiments, the P450 exhibits well-known P450 motifs and/or secondary structure. Generally, P450 sequence identity is determined by alignment of the full amino acid sequences, except for the N-terminal transmembrane regions (e.g., the alignment does not include about the first 30 amino acids of the wild-type sequence). In some embodiments, the engineered P450 enzyme comprises an amino acid sequence that has at least about 60% identity, at least about 70% identity, at least about 75% identity, at least about 80% identity, at least about 85% identity, at least about 90% identity, at least about 95% identity, at least about 96% identity, at least about 97% identity, or at least about 98% identity to any one of SEQ ID NOS: 48 to 60 or any P450 enzymes disclosed in U.S. Application No. 62/635,751, the contents of which are incorporated by reference in its entirety. SEQ ID NOS: 48 to 60 show P450 enzymes without the predicted transmembrane region (txx is the length of the N-terminal truncation): t22ZzHO (SEQ ID NO:48), t20BsGAO (SEQ ID NO:49), t16HmPO (SEQ ID NO:50), t19LsGAO (SEQ ID NO:51), t16NtEAO (SEQ ID NO:52), t26CpVO (SEQ ID NO:53), t23AaAO (SEQ ID NO:54), t21AtKO (SEQ ID NO:55), t30SrKO (SEQ ID NO:56), t52PpKO (SEQ ID NO:57), t15PsVO (SEQ ID NO:58), t20CiVO (SEQ ID NO:59), t20HaGAO (SEQ ID NO:60).

The similarity of nucleotide and amino acid sequences, i.e., the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST polynucleotide searches can be performed with the BLASTN program, score=100, word length=12.

BLAST protein searches may be performed with the BLASTP program, score=50, word length=3. To obtain gapped alignments for comparative purposes, Gapped BLAST is utilized as described in Altschul et al (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

In various embodiments, the engineered P450 enzyme may comprise an amino acid sequence having one or more amino acid mutations relative to the wild-type sequence, not including the modifications to the N-terminal transmembrane region (e.g., about the first 18 to 30 amino acids). For example, the P450 enzyme may comprise an amino acid sequence having from 1 to about 50, or from 1 to about 40, or from 1 to about 30, or from 1 to about 25, or from 1 to about 20, or from 1 to about 15, or from 1 to about 10, or from 1 to about 5 mutations relative to the wild-type sequence (e.g., any one of SEQ ID NOS: 48 to 60). In some embodiments, the one or more amino acid mutations may be independently selected from substitutions, insertions, deletions, and truncations. In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups:

(1) hydrophobic: Met, Ala, Val, Leu, Ile;

(2) neutral hydrophilic: Cys, Ser, Thr; Asn, Gin;

(3) acidic: Asp, Glu;

(4) basic: His, Lys, Arg;

(5) residues that influence chain orientation: Gly, Pro; and

(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gin; (iv) Lys and Arg; and (v) Tyr and Phe.

As used herein, “non-conservative substitutions” are defined as exchanges of an amino acid by another amino acid listed in a different group of the six standard amino acid groups (1) to (6) shown above.

In various embodiments, the engineered P450 enzyme has a deletion or truncation of part or all of it native transmembrane domain. Generally, the deletion or truncation is about the first 15 to 30 amino acids, and the desired length can be determined based on the present disclosure and using predictive tools known in the art (e.g., PHOBIUS, http://phobius.sbc.su.se/). See Lukas K, et al., A Combined Transmembrane Topology and Signal Peptide Prediction Method, Journal of Molecular Biology, 338(5):1027-1036 (2004); Lukas K, et al., An HMM posterior decoder for sequence feature prediction that includes homology information, Bioinformatics, 21 (Suppl 1):i251-i257 (2005); Lukas K, et al., Advantages of combined transmembrane topology and signal peptide prediction—the Phobius web server, Nucleic Acids Res., 35:W429-32 (2007).

In various embodiments, the engineered P450 enzyme may have an N-terminal truncation of from about 10 to about 55 amino acids, or from about 15 to about 45 amino acids, or from about 15 to about 40 amino acids, or from about 15 to about 35 amino acids with respect to the wild-type enzyme. In various embodiments, the engineered P450 enzyme may have an N-terminal truncation of about 10, about 11, about 12, about 13, about 14, about 15, about 16, about 17, about 18, about 19, about 20, about 21, about 22, about 23 about 24, about 25, about 26, about 27, about 28, about 29, about 30, about 31, about 32, about 33, about 34, about 35, about 36, about 37, about 38, about 39, or about 40 amino acids with respect to the wild-type enzyme.

In one aspect, the present technology relates to a P450 enzyme (e.g., any of the P450 enzymes disclosed above, and variants thereof) having a bacterial or yeast transmembrane domain derived from a bacterial or yeast membrane protein, such as an inner membrane protein. In some embodiments, the P450 wild-type transmembrane region is replaced with a membrane anchor sequence derived from a bacterial or yeast protein. In some embodiments, the bacterial or yeast protein is an inner membrane protein with its C-terminus in the cytoplasm. The bacterial or yeast transmembrane domain (or derivative thereof) replaces part or the entire native P450 N-terminal transmembrane region. In some embodiments, the bacterial or yeast transmembrane domain is a single-pass transmembrane domain. In some embodiments, the bacterial or yeast transmembrane domain is a multi-pass (e.g., 2, 3, 4, 5, 6, 7, 8, 9, 10, 11, 12 or more transmembrane helices) transmembrane domain. In some embodiments, the N-terminal anchor sequence is derived from an inner membrane protein of the microbial host genus or species.

In some embodiments, the transmembrane anchor sequence derived from the bacterial or yeast protein is from about 8 to about 75 amino acids in length. For example, in some embodiments, the membrane anchor is from about 15 to about 50, or from about 15 to about 40, or from about 15 to about 30, or from about 20 to about 40, or from about 20 to about 30 amino acids in length. In some embodiments, the membrane anchor is about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, or about 75 amino acids in length.

In some embodiments, the transmembrane anchor sequence derived from the bacterial or yeast protein is from about 50 to about 500 amino acids in length. For example, in some embodiments, the membrane anchor is from about 75 to about 475, or from about 100 to about 450, or from about 125 to about 425, or from about 150 to about 400, or from about 175 to about 375, or from about 200 to about 350, or from about 225 to about 325, or from about 250 to about 300 amino acids in length.

In some embodiments, the bacterial transmembrane domain is derived from Bacillus subtilis (B. subtilis). By way of example, in some embodiments, the B. subtilis transmembrane domain includes, but is not limited to, N-terminal domains from the following genes: ATPL_BACSU, LON2_BACSU, and PTG3C_BACSU. In some embodiments, the transmembrane regions are determined by predictive tools known in the art (including, e.g., PHOBIUS).

In some embodiments, a B. subtilis single-pass transmembrane domain comprises an amino acid sequence of SEQ ID NOS: 61 or 62, or a derivative thereof. In some embodiments, a B. subtilis multi-pass transmembrane domain comprises an amino acid sequence of SEQ ID NO: 63 (12-pass transmembrane domain), or a derivative thereof. The derivative may have one or more amino acid mutations relative to the wild-type B. subtilis sequence. In some embodiments, the transmembrane domain may have from about 1 to about 10, or from about 1 to about 8, or from about 1 to about 5, or from about 1 to about 3 mutations relative to the wild-type B. subtilis sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is B. subtilis.

In some embodiments, the bacterial transmembrane domain is derived from Corynebacterium spp. By way of example, in some embodiments, the Corynebacterium spp. transmembrane domain includes, but is not limited to, N-terminal domains from the following genes: A4QFD0_CORGB, ATPF_CORGL, and COX2_CORGL. In some embodiments, the transmembrane regions are determined by predictive tools known in the art (including, e.g., PHOBIUS).

In some embodiments, a Corynebacterium spp. single-pass transmembrane domain comprises an amino acid sequence of SEQ ID NOS: 64 or 65, or a derivative thereof. In some embodiments, a Corynebacterium spp. multi-pass transmembrane domain comprises an amino acid sequence of SEQ ID NO: 66 (4-pass transmembrane domain), or a derivative thereof. The derivative may have one or more amino acid mutations relative to the wild-type Corynebacterium spp. sequence. In some embodiments, the transmembrane domain may have from about 1 to about 10, or from about 1 to about 8, or from about 1 to about 5, or from about 1 to about 3 mutations relative to the wild-type Corynebacterium spp. sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is Corynebacterium spp.

In some embodiments, the bacterial transmembrane domain is derived from Pseudomonas spp. By way of example, in some embodiments, the Pseudomonas spp. transmembrane domain includes, but is not limited to, N-terminal domains from the following genes: WP_034025480.1, WP_090310142.1, and FTSK_PSEAE. In some embodiments, the transmembrane regions are determined by predictive tools known in the art (including, e.g., PHOBIUS).

In some embodiments, a Pseudomonas spp. single-pass transmembrane domain comprises an amino acid sequence of SEQ ID NOS: 67 or 68, or a derivative thereof. In some embodiments, a Pseudomonas spp. multi-pass transmembrane domain comprises an amino acid sequence of SEQ ID NO: 69 (4-pass transmembrane domain), or a derivative thereof. The derivative may have one or more amino acid mutations relative to the wild-type Pseudomonas spp. sequence. In some embodiments, the transmembrane domain may have from about 1 to about 10, or from about 1 to about 8, or from about 1 to about 5, or from about 1 to about 3 mutations relative to the wild-type Pseudomonas spp. sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is Pseudomonas spp.

In some embodiments, the bacterial transmembrane domain is derived from Rhodobacter spp. By way of example, in some embodiments, the Rhodobacter spp. transmembrane domain includes, but is not limited to, N-terminal domains from the following genes: RCEH_RHOSH, UCRI_RHOCA, and Q9L906_RHOCA. In some embodiments, the transmembrane regions are determined by predictive tools known in the art (including, e.g., PHOBIUS).

In some embodiments, a Rhodobacter spp. single-pass transmembrane domain comprises an amino acid sequence of SEQ ID NOS: 70 or 71, or a derivative thereof. In some embodiments, a Rhodobacter spp. multi-pass transmembrane domain comprises an amino acid sequence of SEQ ID NO: 72 (6-pass transmembrane domain), or a derivative thereof. The derivative may have one or more amino acid mutations relative to the wild-type Rhodobacter spp. sequence. In some embodiments, the transmembrane domain may have from about 1 to about 10, or from about 1 to about 8, or from about 1 to about 5, or from about 1 to about 3 mutations relative to the wild-type Rhodobacter spp. sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is Rhodobacter spp.

In some embodiments, the bacterial transmembrane domain is derived from Vibrio spp. By way of example, in some embodiments, the Vibrio spp. transmembrane domain includes, but is not limited to, N-terminal domains from the following genes: WP_020333352.1, WP_065296230.1, and MSBA_VIBCH. In some embodiments, the transmembrane regions are determined by predictive tools known in the art (including, e.g., PHOBIUS).

In some embodiments, a Vibrio spp. single-pass transmembrane domain comprises an amino acid sequence of SEQ ID NOS: 73 or 74. In some embodiments, a Vibrio spp. multi-pass transmembrane domain comprises an amino acid sequence of SEQ ID NO: 75 (6-pass transmembrane domain), or a derivative thereof. The derivative may have one or more amino acid mutations relative to the wild-type Vibrio spp. sequence. In some embodiments, the transmembrane domain may have from about 1 to about 10, or from about 1 to about 8, or from about 1 to about 5, or from about 1 to about 3 mutations relative to the wild-type Vibrio spp. sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is Vibrio spp.

In some embodiments, the bacterial transmembrane domain is derived from Zymomonas spp. By way of example, in some embodiments, the Zymomonas spp. transmembrane domain includes, but is not limited to, N-terminal domains from the following genes: CCME_ZYMMO, WP_023593463.1, and WP_011240504.1. In some embodiments, the transmembrane regions are determined by predictive tools known in the art (including, e.g., PHOBIUS).

In some embodiments, a Zymomonas spp. single-pass transmembrane domain comprises an amino acid sequence of SEQ ID NOS: 76 or 77, or a derivative thereof. In some embodiments, a Zymomonas spp. multi-pass transmembrane domain comprises an amino acid sequence of SEQ ID NO: 78 (3-pass transmembrane domain), or a derivative thereof. The derivative may have one or more amino acid mutations relative to the wild-type Zymomonas spp. sequence. In some embodiments, the transmembrane domain may have from about 1 to about 10, or from about 1 to about 8, or from about 1 to about 5, or from about 1 to about 3 mutations relative to the wild-type Zymomonas spp. sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is Zymomonas spp.

In some embodiments, the yeast transmembrane domain is derived from Pichia pastoris (P. pastoris). By way of example, in some embodiments, the P. pastoris transmembrane domain includes, but is not limited to, N-terminal domains from the following genes: ANZ73349.1, SEC11_KOMPG, and SHO1_KOMPG. In some embodiments, the transmembrane regions are determined by predictive tools known in the art (including, e.g., PHOBIUS).

In some embodiments, a P. pastoris single-pass transmembrane domain comprises an amino acid sequence of SEQ ID NOS: 79 or 80, or a derivative thereof. In some embodiments, a P. pastoris multi-pass transmembrane domain comprises an amino acid sequence of SEQ ID NO: 81 (4-pass transmembrane domain), or a derivative thereof. The derivative may have one or more amino acid mutations relative to the wild-type P. pastoris sequence. In some embodiments, the transmembrane domain may have from about 1 to about 10, or from about 1 to about 8, or from about 1 to about 5, or from about 1 to about 3 mutations relative to the wild-type P. pastoris sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is P. pastoris.

In some embodiments, the yeast transmembrane domain is derived from Saccharomyces cerevisiae (S. cerevisiae). By way of example, in some embodiments, the S. cerevisiae transmembrane domain includes, but is not limited to, N-terminal domains from the following genes: BCS1_YEAST, YFH6_YEAST, and YB85_YEAST. In some embodiments, the transmembrane regions are determined by predictive tools known in the art (including, e.g., PHOBIUS).

In some embodiments, a S. cerevisiae single-pass transmembrane domain comprises an amino acid sequence of SEQ ID NOS: 82 or 83, or a derivative thereof. In some embodiments, a S. cerevisiae multi-pass transmembrane domain comprises an amino acid sequence of SEQ ID NO: 84 (12-pass transmembrane domain), or a derivative thereof. The derivative may have one or more amino acid mutations relative to the wild-type S. cerevisiae sequence. In some embodiments, the transmembrane domain may have from about 1 to about 10, or from about 1 to about 8, or from about 1 to about 5, or from about 1 to about 3 mutations relative to the wild-type S. cerevisiae sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is S. cerevisiae.

In some embodiments, the yeast transmembrane domain is derived from Yarrowia lipolytica (Y lipolytica). By way of example, in some embodiments, the Y lipolytica transmembrane domain includes, but is not limited to, N-terminal domains from the following genes: XP_502343.1, XP_502366.1, and XP_500492.1. In some embodiments, the transmembrane regions are determined by predictive tools known in the art (including, e.g., PHOBIUS).

In some embodiments, a Y lipolytica single-pass transmembrane domain comprises an amino acid sequence of SEQ ID NOS: 85 or 86, or a derivative thereof. In some embodiments, a Y lipolytica multi-pass transmembrane domain comprises an amino acid sequence of SEQ ID NO: 87 (12-pass transmembrane domain), or a derivative thereof. The derivative may have one or more amino acid mutations relative to the wild-type Y lipolytica sequence. In some embodiments, the transmembrane domain may have from about 1 to about 10, or from about 1 to about 8, or from about 1 to about 5, or from about 1 to about 3 mutations relative to the wild-type Y lipolytica sequence. In some embodiments, the one or more amino acid mutations may be independently selected from substitutions, insertions, and deletions. In some embodiments, the amino acid mutations are amino acid substitutions. In some embodiments, mutations are selected based on their predicted score as a transmembrane region, using known predictive tools. In some embodiments, the microbial host is Y. lipolytica.

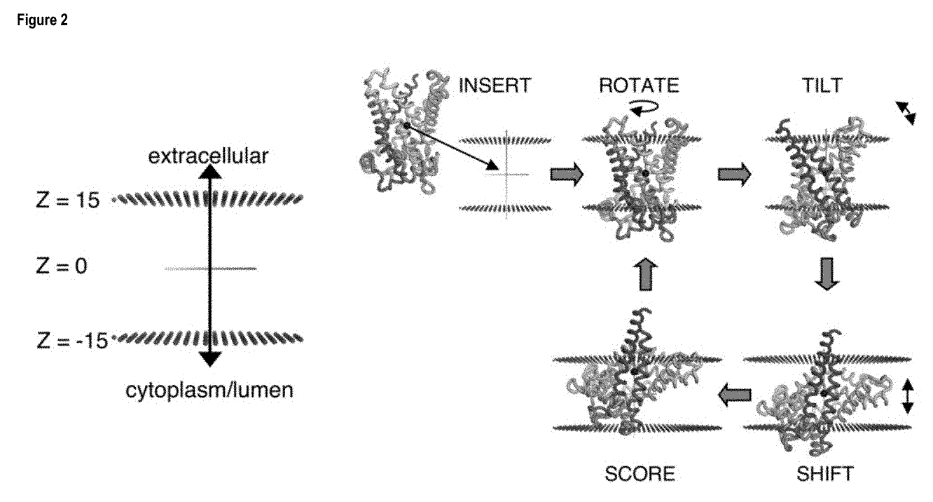

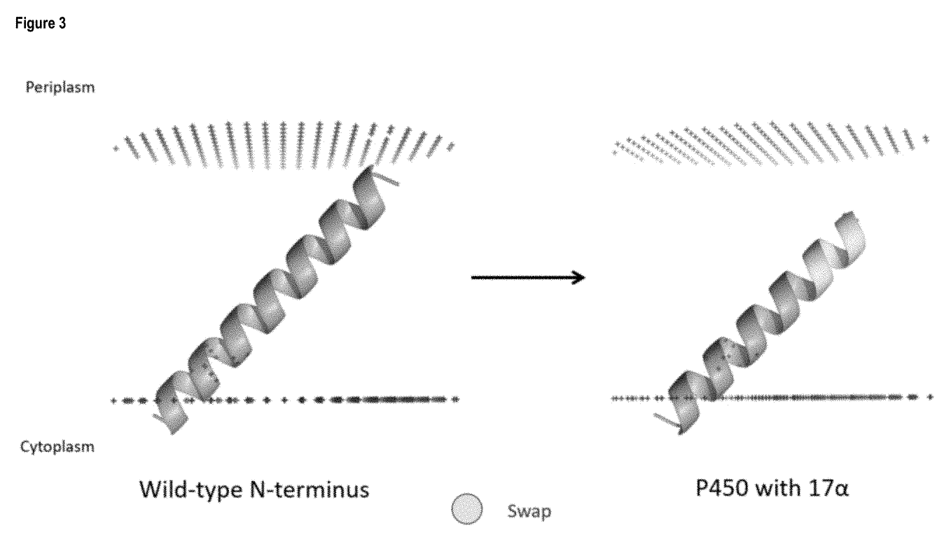

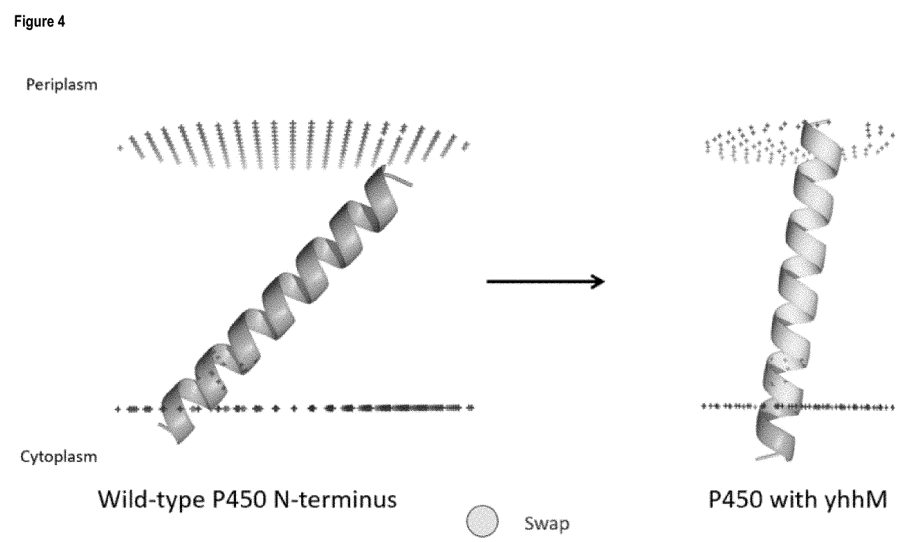

In some embodiments, the effect of different N-terminal sequences on anchoring P450's to the membrane, e.g., the inner membrane, is conducted in silico by computer modeling or computer simulation or by computational methods. By way of example, in some embodiments, the E(z) potential, or similar predictive tool, is used to determine the effects different N-terminal sequences have on anchoring P450's to the membrane by modeling the three dimensional depth and tilt of the sequence in the membrane. See FIG. 2. By way of example, that E(z) potential predicts that wild type valencene oxygenase (VO), an enzyme derived from Stevia rebaudiana Kaurene Oxidase (SrKO), and VO fused to the modified N-terminus bovine 17α membrane anchor will not span the membrane. See FIG. 3. By way of example, the E(z) potential predicts that VO fused to an N-terminal anchor of E. coli yyhm transmembrane anchor will span the membrane. See FIG. 4. Without wishing to be bound by theory, in some embodiments, P450 enzymes with N-terminal transmembrane proteins that span the entire membrane result in P450 enzymes with improved anchoring and improved function.

In other aspects, the present technology provides polynucleotides comprising a nucleotide sequence encoding any of the engineered P450 enzymes described above. In some embodiments, the polynucleotide includes a codon optimized for expression in bacteria or yeast cells. In some embodiments, the polynucleotide also comprises a nucleotide sequence encoding at least one engineered P450 enzyme with one or more cytochrome P450 reductase (CPR) enzymes described herein as a translational fusion or operon. Such polynucleotides may further comprise, in addition to sequences encoding the engineered P450 enzyme, one or more expression control elements. For example, the polynucleotide may comprise one or more promoters or transcriptional enhancers, ribosomal binding sites, transcription termination signals, as expression control elements. The polynucleotide may be inserted within any suitable vector, including an expression vector, and which may be contained within any suitable host cell for expression. The polynucleotide may be designed for introduction and/or protein expression in any suitable host cell, including bacterial and yeast cells.

In other aspects, the present technology provides bacteria or yeast host cells expressing the engineered P450 enzyme, either integrated into the genome, or extrachromosomally (e.g., on a plasmid). In some embodiments, the P450 enzyme is expressed by a strong promoter, such as T7, T5, T3, or Trc, or a promoter having promoter strength in bacteria or yeast equal to or more than T7, T5, T3, or Trc. The promoter may be a strong constitutive bacteria or yeast promoter or a coliphage promoter, or a variant thereof. Deuschle et al., Promoters of Escherichia coli: a hierarchy of in vivo strength indicates alternate structures, EMBO J. 5(11): 2987-2994 (1986); http://parts.igem.org/Promoters/Catalog/Ecoli/Constitutive.

In some embodiments, when expressed from a plasmid, the plasmid may be a low or high copy number plasmid (e.g., p5, p10, p20). In some embodiments, the P450 gene is chromosomally integrated into the genome of the bacteria or yeast host cell, which may further include tandem repeats of the gene to increase the expression level. By way of example, see US 20110236927, which is hereby incorporated by reference in its entirety.

In some embodiments, the bacteria or yeast cells are fed the desired P450 substrate for chemical transformation, or biochemical pathways may be expressed in the bacteria or yeast host cell to generate the substrate in vivo.

In some embodiments the bacteria or yeast host cell expresses one or more recombinant biosynthetic pathways. For example, a bacteria or yeast host cell may express one or more recombinant biosynthetic pathways that include at least 1, at least 2, at least 3, at least 4, or at least 5 recombinant enzymes. The biosynthetic pathways may produce a secondary metabolite through the overexpression of at least 1, at least 2, at least 3, at least 4, or at least 5 foreign genes. In these or other embodiments, the bacteria or yeast host cell may overexpress at least 1, at least 2, at least 3, at least 4, or at least 5 native bacteria or yeast genes. Overexpression of several bacteria or yeast genes and/or foreign genes can produce substantial cell stress responses. Where these pathways include one or more P450 enzymes, these stress responses can be substantially higher. For example, as shown herein, P450 enzymes can induce substantial overexpression of the IbpA protein, a protein that is overexpressed under conditions of protein aggregation and cell stress. For example, overexpression of native bacteria or yeast genes as well as foreign genes can result in conditions of protein aggregation that induce a cell stress response (e.g., as observed by overexpression of IbpA).

In various embodiments, the present technology results in reduced cell stress such that the bacteria or yeast host cell does not exhibit a substantially stressed phenotype during culturing, or the cell stress is minimized. Cell stress may be assessed by measuring the expression of various cell stress proteins including, but not limited to, IbpA, DnaK, GrpE, and GroL. In some embodiments, methods of the present technology do not result in overexpression of the cell stress protein IbpA. In this context, overexpression refers to at least two times the IbpA expression level of the parent strain. In some embodiments, the engineering of the P450 enzyme of the present technology may be guided by testing IbpA expression in cultures. For example, a determination of the length of the truncation of the P450 enzyme and/or the anchor size and/or sequence may be guided by IbpA expression levels in the culture.

In some embodiments, at least one foreign gene is expressed by a strong promoter, such as T7, T5, T3, or Trc, or a promoter having promoter strength in bacteria or yeast cells equal to or more than T7, T5, T3, or Trc. In some embodiments, the promoter may be a strong constitutive bacteria or yeast promoter or a coliphage promoter, or a variant thereof. Deuschle et al., Promoters of Escherichia coli: a hierarchy of in vivo strength indicates alternate structures, EMBO J. 5(11): 2987-2994 (1986); http://parts.igem.org/Promoters/Catalog/Ecoli/Constitutive. In an embodiment, the genes are expressed from a plasmid, which may be a low or high copy number plasmid (e.g., p5, p10, p20). In another embodiment, the genes are chromosomally integrated into the genome of the bacteria or yeast host cell, which may further include tandem repeats of the gene to increase the expression level. See US 20110236927, which is hereby incorporated by reference in its entirety.

In some embodiments, the bacteria or yeast host cell produces a compound from isopentenyl pyrophosphate (IPP) and/or dimethylallyl pyrophosphate DMAPP, such as a terpene or terpenoid compound. In an exemplary embodiment, the bacteria or yeast cell may overexpress at least one gene in the MEP pathway, which is endogenous to the bacteria or yeast cell. The MEP (2-C-methyl-D-erythritol 4-phosphate) pathway, also called the MEP/DOXP (2-C-methyl-D-erythritol 4-phosphate/I-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. In the MEP pathway, pyruvate and D-glyceraldehyde-3-phosphate are converted via a series of reactions to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, ispA, and ispB. In some embodiments, one or more terpenoid compounds are produced at least in part by metabolic flux through an MEP pathway. In an embodiment, the bacteria or yeast host cell may express at least one additional copy of a dxs and/or idi gene. In some embodiments, the bacteria or yeast host as at least one additional copy of dxs, ispD, ispF, and/or idi gene, so as to overexpress these gene products.

In various embodiments, the bacteria or yeast host cell expresses one or more biosynthetic pathways that include at least one membrane-anchored engineered P450 enzyme as described herein. In some embodiments, the P450 enzyme is not strongly expressed, but the more efficient membrane anchoring in accordance with the present technology allows for sufficient activity without stronger expression. In various embodiments, the bacteria or yeast host cell expresses at least 1, at least 2, at least 3, at least 4, or at least 5 P450 enzymes, which may operate in serial fashion in a biosynthetic pathway.

In some embodiments, the P450 enzyme is expressed from a strong (e.g., constitutive or inducible) bacteria or yeast promotor or coliphage promoter, or variant thereof (e.g., Trc, T7, T5, or T3, or variant thereof). While overexpression of P450 enzymes can induce significant cell stress, the membrane anchoring system in accordance with the present technology renders the P450-membrane association more productive, with less protein misfolding and/or aggregation, which would otherwise induce cell stress.

In various embodiments, the bacteria or yeast host cell expresses the engineered P450 enzyme alongside one or more cytochrome P450 reductase (CPR) partner that regenerates the P450 enzyme. As used herein, the term “cytochrome P450 reductase partner” or “CPR partner” refers to a cytochrome P450 reductase capable of regenerating the cofactor component of the cytochrome P450 oxidase of interest for oxidative chemistry. The CPR may be a natural CPR partner for the P450 enzyme, and in other embodiments, the CPR partner is not the natural CPR partner for the P450 enzyme. In nature, cytochrome P450 reductase is a membrane protein generally found in the endoplasmic reticulum. It catalyzes pyridine nucleotide dehydration and electron transfer to membrane bound cytochrome P450s. In some embodiments, CPRs are derived from any species that naturally employs P450 biochemistry, including, for example: Zingiber sp., Barnadesia sp., Hyoscyamus sp., Latuca sp., Nicotiana sp., Citrus sp., Artemesia sp., Arabidopsis sp, Stevia sp., Siraitia sp., Bacillus sp., Pleurotus sp., Cichorium sp., Helianthus sp., and Physcomitrella sp., Taxus sp., Rosa sp., Cymbopogon sp., Humulus sp., Pogostemon sp., and Cannabis sp. Exemplary CPRs include those from Stevia rebaudiana (e.g., SEQ ID NO: 33, 40, 41, and 42), Arabidopsis thaliana (SEQ ID NO: 34, 37, 38, and 39), Taxus cuspidata (SEQ ID NO: 35), Atemisia annua (SEQ ID NO:36), and Pelargonium graveolans (SEQ ID NO: 43). In various embodiments, the wild-type CPR or derivative thereof is expressed separately from the P450 enzymes (e.g., from the same or different operon), or in some embodiments as a translational fusion with the P450 enzyme. In some embodiments, the CPR enzyme or derivative thereof may be based on sequences of CPR enzymes discloses in U.S. Application No. 62/635,751, the contents of which are incorporated by reference in its entirety. Generally, CPR derivatives comprise amino acid sequences having at least 70%, or at least 80%, or at least 90%, or at least 95% identity to the wild-type sequence (e.g., SEQ ID NOS: 33-43), and which can be employed in the various embodiments.

In an embodiment, the CPR may be expressed as a translational fusion protein with an engineered P450 enzyme. The CPR may be fused to the P450 enzyme through a linker. Exemplary linker sequences can be predominantly serine, glycine, and/or alanine, and may be from three to one hundred amino acids in various embodiments. Linker sequences include, for example, GSG, GSGGGGS (SEQ ID NO: 44), GSGEAAAK (SEQ ID NO: 45), GSGEAAAKEAAAK (SEQ ID NO: 46), and GSGMGSSSN (SEQ ID NO: 47).

In some embodiments, the present technology allows for better control of P450 enzyme efficiency, by allowing for efficient ratios of expression of P450 enzymes in relation to the CPR partner (when expressed separately). In some embodiments, the ratio of the expression levels of the P450 enzyme(s) and the CPR partners may range from about 5:1 to about 1:5, for example, about 5:1, or about 4:1, or about 3:1, or about 2:1, or about 1:1, or about 1:2, or about 1:3, or about 1:4, or about 1:5. For example, the ratio of the expression levels of the P450 enzyme(s) and the CPR partner may be from about 2:1 to about 1:2. In various embodiments, the CPR may also be modified to include at least one membrane anchor sequence derived from a bacteria or yeast protein as described herein. In an embodiment, the bacteria or yeast host cell expresses a single CPR protein, and optionally expresses more than one P450 enzyme.

In various embodiments, the bacteria or yeast host cell expresses a biosynthetic pathway that produces a secondary metabolite selected from a terpenoid, alkaloid, cannabinoid, steroid, saponin, glycoside, stilbenoid, polyphenol, antibiotic, polyketide, fatty acid, or non-ribosomal peptide. In certain embodiments, the bacteria or yeast host cell produces one or more terpenoid compounds. A terpenoid, also referred to as an isoprenoid, is an organic chemical derived from a five-carbon isoprene unit (C5). Several non-limiting examples of terpenoids, classified based on the number of isoprene units that they contain, include: hemiterpenoids (1 isoprene unit), monoterpenoids (2 isoprene units), sesquiterpenoids (3 isoprene units), diterpenoids (4 isoprene units), sesterterpenoids (5 isoprene units), triterpenoids (6 isoprene units), tetraterpenoids (8 isoprene units), and polyterpenoids with a larger number of isoprene units. In an embodiment, the bacteria or yeast host cell produces a terpenoid selected from a monoterpenoid, a sesquiterpenoid, diterpenoid, a sesterpenoid, or a triterpenoid. Terpenoids represent a diverse class of molecules that provide numerous commercial applications, including in the food and beverage industries as well as the perfume, cosmetic and health care industries. By way of example, terpenoid compounds find use in perfumery (e.g. patchoulol), in the flavor industry (e.g., nootkatone), as sweeteners (e.g., steviol), or therapeutic agents (e.g., taxol) and many are conventionally extracted from plants. Nevertheless, terpenoid molecules are found in ppm levels in nature, and therefore require massive harvesting to obtain sufficient amounts for commercial applications.

Where the chemical species is a terpenoid, the host cell will generally contain a recombinant downstream pathway that produces the terpenoid from IPP and DMAPP precursors. Terpenes such as Monoterpenes (010), Triterpenoids, Sesquiterpenes (C15) and Diterpenes (C20) are derived from the prenyl diphosphate substrates, geranyl diphosphate (GPP), farnesyl diphosphate (FPP) and geranylgeranyl diphosphate (GGPP) respectively through the action of a very large group of enzymes called the terpene (terpenoid) synthases. These enzymes are often referred to as terpene cyclases since the product of the reactions are cyclized to various monoterpene, sesquiterpene and diterpene carbon skeleton products. Many of the resulting carbon skeletons undergo subsequence oxygenation by cytochrome P450 hydrolysase enzymes to give rise to large families of derivatives. In various embodiments, the bacteria or yeast cell expresses a biosynthetic pathway involving the overexpression of a geranyl diphosphate synthase (GPS), a gernanylgeranyl diphosphate synthase (GGPS), a farnsesyl diphosphate synthase (FPS), or a farnesyl geranyl diphosphate synthase (FGPPS).

In some embodiments, the product of the present technology, is one or more oxygenated terpenoids. As used herein, the term “oxygenated terpenoid” refers to a terpene scaffold having one or more oxygenation events, producing a corresponding alcohol, aldehyde, carboxylic acid and/or ketone. In some embodiments, the bacteria or yeast host cell produces at least one terpenoid selected from alpha-sinensal, beta-Thujone, Camphor, Carveol, Carvone, Cineole, Citral, Citronellal, Cubebol, Geraniol, Limonene, Menthol, Menthone, Myrcene, Nootkatone, Nootkatol, Patchouli, Piperitone, Sabinene, Steviol, Steviol glycoside, Taxadiene, Thymol, and Valencene, either as a P450 oxygenated product or as a substrate for P450 chemistry.

In another embodiment, the bacteria or yeast host cell produces Valencene and/or Nootkatone. In such an embodiment, the bacteria or yeast host cell may express a biosynthetic pathway that further includes a farnesyl pyrophosphate synthase, a Valencene Synthase, and a Valencene Oxidase (the VO comprising the membrane anchor described herein). Farnesyl pyrophosphate synthases (FPPS) produce farnesyl pyrophosphates from iso-pentyl pyrophosphate (IPP) and dimethylallyl pyrophosphate (DMAPP). An exemplary farnesyl pyrophosphate synthase is ERG20 of Saccharomyces cerevisiae (NCBI accession P08524) and E. coli ispA. Valencene synthase produces sesquiterpene scaffolds and are described in, for example, US 2012/0107893, US 2012/0246767, and U.S. Pat. No. 7,273,735, which are hereby incorporated by reference in their entireties.

In an embodiment, the bacteria or yeast host cell produces steviol or steviol glycoside (e.g., RebM). Steviol is produced from kaurene by the action of two P450 enzymes, kaurene oxidase (KO) and kaurenoic acid hydroxylase (KAH). After production of steviol, various steviol glycoside products may be produced through a series of glycosylation reactions, which can take place in vitro or in vivo. Pathways and enzymes for production of steviol and steviol glycosides are disclosed in US 2013/0171328, US 2012/0107893, WO 2012/075030, WO 2014/122328, which are hereby incorporated by reference in their entireties.

In various embodiments, the bacteria or yeast host cell may express a biosynthetic pathway involving a geranylgeranyl pyrophosphate synthase (GPPS), a copalyl diphosphate synthase (CPPS), and a kaurene synthase (KS), as well as a kaurene oxidase (KO) and a kaureneoic acid hydroxylase (KAH) having a transmembrane domain derived from a bacteria or yeast gene. In some embodiments, the biosynthetic pathway may further include or more uridine diphosphate dependent glycosyltransferase enzymes (UGT).

Other biosynthetic pathways for production of terpenoid compounds are disclosed in U.S. Pat. No. 8,927,241, which is hereby incorporated by reference in its entirety.

In various embodiments, bacteria or yeast host cell is cultured to produce the one or more chemical species. For example, the bacteria or yeast host cell may be cultured to produce one or more terpenoid compounds.

While commercial biosynthesis in bacteria or yeast host cells can be limited by the temperature at which overexpressed and/or foreign enzymes are stable, the substantial improvements in stability of the P450 enzymes described herein, may allow for cultures to be maintained at higher temperatures, resulting in higher yields and higher overall productivity. In some embodiments, the culturing is conducted at about 30° C. or greater, about 31° C. or greater, about 32° C. or greater, about 33° C. or greater, about 34° C. or greater, about 35° C. or greater, about 36° C. or greater, or about 37° C. or greater.

In some embodiments, the bacteria or yeast host cells are further suitable for commercial production of chemical species, and therefor can be productive at commercial scale. In some embodiments, the size of the culture is at least about 100 L, at least about 200 L, at least about 500 L, at least about 1,000 L, or at least about 10,000 L. In an embodiment, the culturing may be conducted in batch culture, continuous culture, or semi-continuous culture.

In various embodiments, methods of the present technology further include recovering the one or more chemical species such as one or more terpenoid compounds from the cell culture or from cell lysates. In some embodiments, the culture produces at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, at least about 100 mg/L, at least about 150 mg/L, or at least about 200 mg/L of the chemical species.

In some embodiments involving the production of a terpenoid compound, the production of indole is used as a surrogate marker for terpenoid production, and/or the accumulation of indole in the culture is controlled to increase terpenoid production. For example, in various embodiments, accumulation of indole in the culture is controlled to below about 100 mg/L, or below about 75 mg/L, or below about 50 mg/L, or below about 25 mg/L, or below about 10 mg/L. The accumulation of indole can be controlled by balancing protein expression and activity using the multivariate modular approach as described in U.S. Pat. No. 8,927,241 (which is hereby incorporated by reference), and/or is controlled by chemical means.

The oxidized product can be recovered by any suitable process, including partitioning the desired product into an organic phase. The production of the desired product can be determined and/or quantified, for example, by gas chromatography (e.g., GC-MS). The desired product can be produced in batch or continuous bioreactor systems. Production of product, recovery, and/or analysis of the product can be done as described in US 2012/0246767, which is hereby incorporated by reference in its entirety. For example, in some embodiments, particularly in relation to terpenoids, oxidized oil is extracted from aqueous reaction medium using an organic solvent, such as an alkane such as heptane, followed by fractional distillation. Terpenoid components of fractions may be measured quantitatively by GC/MS, followed by blending of fractions to generate a desired product profile.

In various embodiments, the recovered chemical species such as one or more terpenoid compounds are incorporated into a product (e.g., a consumer or industrial product). For example, the product may be a flavor product, a fragrance product, a sweetener, a cosmetic, a cleaning product, a detergent or soap, or a pest control product. In some embodiments, the oxygenated product recovered is nootkatol and/or nootkatone, and the product is a flavor product selected from a beverage, a chewing gum, a candy, or a flavor additive, or the product is an insect repellant. In some embodiments, the oxygenated product is steviol or a steviol glycoside, which is provided as a sweetener, or is incorporated into beverages or food products.

The present technology further provides methods of making products such as foods, beverages, texturants (e.g., starches, fibers, gums, fats and fat mimetics, and emulsifiers), pharmaceutical products, tobacco products, nutraceutical products, oral hygiene products, and cosmetic products, by incorporating the chemical species produced herein. The higher yields of such species produced in embodiments of the present technology can provide significant cost advantages as well as sustainability.

By way of non-limiting example, in some embodiments, a bacterial host cell of the present technology described herein is selected from B. subtilis, Corynebacterium spp., Pseudomonas spp., Rhodobacterspp., Vibrio spp., and Zymomonas spp.

By way of non-limiting example, in some embodiments, a yeast host cell of the present technology described herein is selected from Pichia pastoris, Saccharomyces cerevisiae, and Yarrowia lipolytica.

### EXAMPLES

### Example 1. Identification of Candidate Bacterial or Yeast Genes with Membrane Anchor Sequences

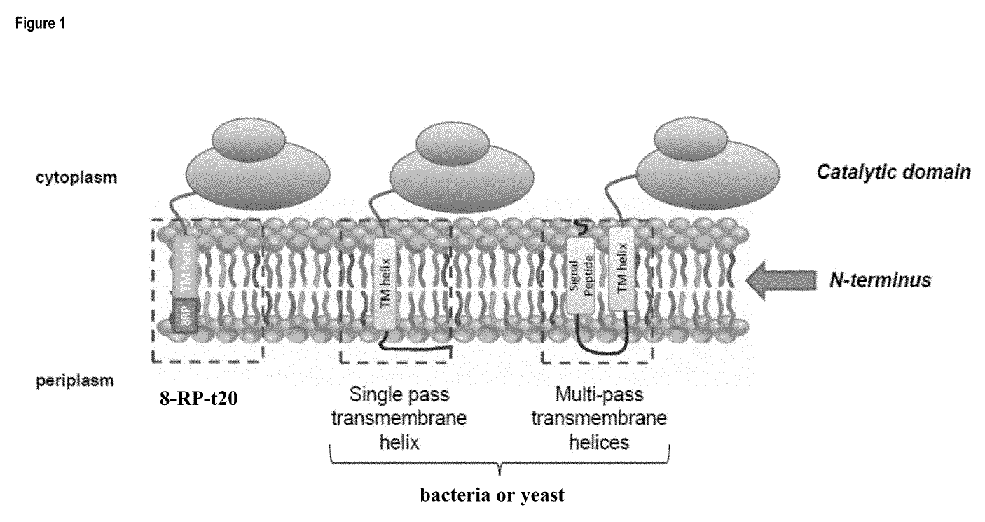

FIG. 1 illustrates the N-terminal membrane anchor concept. Typically, P450 enzymes are expressed in E. coli by truncation of at least a portion of the P450 N-terminal transmembrane region, with the addition of an 8 amino acid peptide (MALLLAVF) derived from bovine P450 17α. See, for example, Barnes H J, et al., Expression and enzymatic activity of recombinant cytochrome P450 17α-hydroxylase in Escherichia coli, PNAS 88: 5597-5601 (1991); and Ajikumar P K, et al., Isoprenoid pathway optimization for taxol precursor overproduction in Escherichia coli, Science 330(6000): 70-74 (2010). However, activity of these P450 constructs in E. coli were generally limiting. In part, the lack of P450 productivity in E. coli was a result from a cell stress response triggered by the non-native membrane anchoring. By engineering P450 proteins to contain an N-terminal anchor derived from a microbial protein that is natively anchored in the inner membrane by its N-terminus (either by single pass or multi-pass transmembrane helices), this cell stress response could be minimized, and other benefits for the functional expression of P450 enzymes might be identified.

To overcome the lack of biophysical understanding, statistical approaches have been applied to position proteins in the membrane (Schramm, et al., Structure, 20(5): 924-35 (2012)). The knowledge based Ez potential (Senes, et al., J. Mol. Biol., 366: 436-448 (2007)) uses an inverse Boltzmann potential (statistical potential or log-odds score; Saven, J. Chem. Phys., 118: 6133-6136 (2003)) to convert observed depth (z coordinate) propensities into amino acid pseudo-energies. FIG. 2 shows the use of E(z) potential to estimate the effect of different N-terminal sequences on anchoring P450's to the membrane, including modeling the three dimensional depth and tilt of the sequence in the membrane. FIG. 3 shows that E(z) potential predicts that a wild type P450 N-terminus and a modified wild type P450 N-terminus, i.e., having a truncated N-terminus that is fused to the modified N-terminus bovine 17α membrane anchor, will not span the membrane. FIG. 4 shows that E(z) potential predicts that a modified wild type P450 N-terminus, i.e., having a truncated N-terminus that is fused to an N-terminal anchor of E. coli yhhM membrane anchor will span the membrane.

SEQUENCES

P450 ENZYMES
>ZzHO [Zingiber zerumbet]
MEAISLFSPFFFITLFLGFFITLLIKRSSRSSVHKQQVLLASLPPSPPRLPLIGNIHQLVGGNPHRILLQLARTHGPLICLRL
GQVDQWASSVEAVEEIIKRHDLKFADRPRDLIFSRIFFYDGNAVVMTPYGGEWKQMRKIYAMELLNSRRVKSFAAIR
EDVARKLTGEIAHKAFAQTPVINLSEMVMSMINAIVIRVAFGDKCKQQAYFLHLVKEAMSYVSSFSVADMYPSLKFLDT
LTGLKSKLEGVHGKLDKVFDEIIAQRQAALAAEQAEEDLIIDVLLKLKDEGNQEFPITYTSVKAIVMEIFLAGTETSSSVID
WVMSELIKNPKAMEKVQKEMREAMQGKTKLEESDIPKFSYLNLVIKETLRLHPPGPLLFPRECRETCEVMGYRVPAG
ARLLINAFALSRDEKYWGSDAESFKPERFEGISVDFKGSNFEFMPFGAGRRICPGMTFGISSVEVALAHLLFHFDWQL
PQGMKIEDLDMMEVSGMSATRRSPLLVLAKLIIPLP (SEQ ID NO: 1)
>BsGAO [Barnadesia spinosa]
MELTLITSLGLAVFVFILFKLLTGSKSTKNSLPEAWRLPIIGHMHHLVGTLPHRGVTDMARKYGSLMHLQLGEVSTIVV
SSPRWAKEVLTTYDITFANRPETLTGEIVAYHNTDIVLSPYGEYWRQLRKLCTLELLSAKKVKSFQSLREEECWNLVK
EVRSSGSGSPVDLSESIFKLIATILSRAAFGKGIKDQREFTEIVKEILRLTGGFDVADIFPSKKILHHLSGKRAKLTNIHNK
LDSLINNIVSEHPGSRTSSSQESLLDVLLRLKDSAELPLTSDNVKAVILDMFGAGTDTSSATIEWAISELIRCPRAMEKV
QTELRQALNGKERIQEEDIQELSYLKLVIKETLRLHPPLPLVMPRECREPCVLAGYEIPTKTKLIVNVFAINRDPEYWKD
AETFMPERFENSPINIMGSEYEYLPFGAGRRMCPGAALGLANVELPLAHILYYFNWKLPNGARLDELDMSECFGATV
QRKSELLLVPTAYKTANNSA (SEQ ID NO: 2)
>HmPO [Hyoscyamus muticus]
MQFFSLVSIFLFLSFLFLLRKWKNSNSQSKKLPPGPWKLPLLGSMLHMVGGLPHHVLRDLAKKYGPLMHLQLGEVSA
VVVISPDMAKEVLKTHDIAFASRPKLLAPEIVCYNRSDIAFCPYGDYWRQMRKICVLEVLSAKNVRSFSSIRRDEVLRL
VNFVRSSTSEPVNFTERLFLFTSSMTCRSAFGKVFKEQETFIQLIKEVIGLAGGFDVADIFPSLKFLHVLTGMEGKIMKA
HHKVDAIVEDVINEHKKNLAMGKTNGALGGEDLIDVLLRLMNDGGLQFPITNDNIKAIIFDMFAAGTETSSSTLVWAMV
QMMRNPTILAKAQAEVREAFKGKETFDENDVEELKYLKLVIKETLRLHPPVPLLVPRECREETEINGYTIPVKTKVMVN
VWALGRDPKYWDDADNFKPERFEQCSVDFIGNNFEYLPFGGGRRICPGISFGLANVYLPLAQLLYHFDWKLPTGME
PKDLDLTELVGVTAARKSDLMLVATPYQPSRE (SEQ ID NO: 3)
>LsGAO [Lactuca sativa]
MELSITTSIALATIVFFLYKLATRPKSTKKQLPEASRLPIIGHMHHLIGTMPHRGVMDLARKHGSLMHLQLGEVSTIVVSS
PKWAKEILTTYDITFANRPETLIGEIIAYHNTDIVLAPYGEYWRQLRKLCTLELLSVKKVKSFQSIREEECWNLVKEVKE
SGSGKPINLSESIFTMIATILSRAAFGKGIKDQREFTEIVKEILRQTGGFDVADIFPSKKFLHHLSGKRARLTSIHKKLDNL
INNIVAEHHVSTSSKANETLLDVLLRLKDSAEFPLTADNVKAIILDMFGAGTDTSSATVEWAISELIRCPRAMEKVQAEL
RQALNGKEKIQEEDIQDLAYLNLVIRETLRLHPPLPLVMPRECREPVNLAGYEIANKTKLIVNVFAINRDPEYWKDAEAF
IPERFENNPNNIMGADYEYLPFGAGRRMCPGAALGLANVQLPLANILYHFNWKLPNGASHDQLDMTESFGATVQRKT
ELLLVPSF (SEQ ID NO: 4)
>NtEAO [Nicotiani tabacum]
MQFFSLVSIFLFLSFLFLLRKWKNSNSQSKKLPPGPWKIPILGSMLHMIGGEPHHVLRDLAKKYGPLMHLQLGEISAVV
VISRDMAKEVLKTHDVVFASRPKIVAMDIICYNQSDIAFSPYGDHWRQMRKICVMELLNAKNVRSFSSIRRDEVVRLID
SIRSDSSSGELVNFTQRIIWFASSMTCRSAFGQVLKGQDIFAKKIREVIGLAEGFDVVDIFPTYKFLHVLSGMKRKLLNA
HLKVDAIVEDVINEHKKNLAAGKSNGALGGEDLIDVLLRLMNDTSLQFPITNDNIKAVIVDMFAAGTETSSTTTVWAMA
EMMKNPSVFTKAQAEVREAFRDKVSFDENDVEELKYLKLVIKETLRLHPPSPLLVPRECREDTDINGYTIPAKTKVMV
NVWALGRDPKYWDDAESFKPERFEQCSVDFFGNNFEFLPFGGGRRICPGMSFGLANLYLPLAQLLYHFDWKLPTGI
MPRDLDLTELSGITIARKGGLYLNATPYQPSRE (SEQ ID NO: 5)
>CpVO [Citrus x paradisi]
MELPLKSIALTIVIVTVLTWAWRVLNWVWLRPKKLEKFLRQQGLKGNSYRLLFGDLKENSIELKEAKARPLSLDDDIAIR
VNPFLHKLVNDYGKNSFMWFGPTPRVNIMNPDQIKAIFTKINDFQKVNSIPLARLLIVGLATLEGEKWAKHRKLINPAFH
QEKLKLMLPAFYLSCIEIITKWEKQMSVEGSSELDVWPYLANLTSDVISRTAFGSSYEEGRRIFQLQAELAELTMQVFR
SVHIPGWRFLPTKRNRRMKEIDKEIRASLMGIIKNREKAMRAGEAANNDLLGILMETSFREIEEHGNNKNVGFSMNDVI
EECKLFYFAGQETTSVLLNWTMVLLSKHQDWQERARQEVLQVFGNNKPDYDGLNHLKIVQMILYEVLRLYPPVTVLS
RAVFKETKLGNLTLPAGVQIGLPMILVHQDPELWGDDAVEFKPERFAEGISKAAKNQVSYFPFALGPRICVGQNFALV
EAKMATAMILQNYSFELSPSYVHAPTAVPILHPELGTQLILRKLWCKNN (SEQ ID NO: 6)
>AaAO [Artemesia annua]
MKSILKAMALSLTTSIALATILLFVYKFATRSKSTKKSLPEPWRLPIIGHMHHLIGTTPHRGVRDLARKYGSLMHLQLGE
VPTIVVSSPKWAKEILTTYDITFANRPETLTGEIVLYHNTDVVLAPYGEYWRQLRKICTLELLSVKKVKSFQSLREEECW
NLVQEIKASGSGRPVNLSENVFKLIATILSRAAFGKGIKDQKELTEIVKEILRQTGGFDVADIFPSKKFLHHLSGKRARLT
SLRKKIDNLIDNLVAEHTVNTSSKTNETLLDVLLRLKDSAEFPLTSDNIKAIILDMFGAGTDTSSSTIEWAISELIKCPKAM
EKVQAELRKALNGKEKIHEEDIQELSYLNMVIKETLRLHPPLPLVLPRECRQPVNLAGYNIPNKTKLIVNVFAINRDPEY
WKDAEAFIPERFENSSATVMGAEYEYLPFGAGRRMCPGAALGLANVQLPLANILYHFNWKLPNGVSYDQIDMTESSG
ATMQRKTELLLVPSF (SEQ ID NO: 7)
>AtKO [Arabidopsis thaliana]
MAFFSMISILLGFVISSFIFIFFFKKLLSFSRKNMSEVSTLPSVPVVPGFPVIGNLLQLKEKKPHKTFTRWSEIYGPIYSIK
MGSSSLIVLNSTETAKEAMVTRFSSISTRKLSNALTVLTCDKSMVATSDYDDFHKLVKRCLLNGLLGANAQKRKRHYR
DALIENVSSKLHAHARDHPQEPVNFRAIFEHELFGVALKQAFGKDVESIYVKELGVTLSKDEIFKVLVHDMMEGAIDVD
WRDFFPYLKWIPNKSFEARIQQKHKRRLAVMNALIQDRLKQNGSESDDDCYLNFLMSEAKTLIKEQIAILVWETIIETA
DTTLVTTEWAIYELAKHPSVQDRLCKEIQNVCGGEKFKEEQLSQVPYLNGVFHETLRKYSPAPLVPIRYAHEDTQIGG
YHVPAGSEIAINIYGCNMDKKRWERPEDWWPERFLDDGKYETSDLHKTMAFGAGKRVCAGALQASLMAGIAIGRLV
QEFEWKLRDGEEENVDTYGLTSQKLYPLMAIINPRRS (SEQ ID NO: 8)
>SrKO [Stevia rebaudiana]
MDAVTGLLTVPATAITIGGTAVALAVALIFWYLKSYTSARRSQSNHLPRVPEVPGVPLLGNLLQLKEKKPYMTFTRWAA
TYGPIYSIKTGATSMVVVSSNEIAKEALVTRFQSISTRNLSKALKVLTADKTMVAMSDYDDYHKTVKRHILTAVLGPNA
QKKHRIHRDIMMDNISTQLHEFVKNNPEQEEVDLRKIFQSELFGLAMRQALGKDVESLYVEDLKITMNRDEIFQVLVVD
PMMGAIDVDWRDFFPYLKWVPNKKFENTIQQMYIRREAVMKSLIKEHKKRIASGEKLNSYIDYLLSEAQTLTDQQLLM
SLWEPIIESSDTTMVTTEWAMYELAKNPKLQDRLYRDIKSVCGSEKITEEHLSQLPYITAIFHETLRRHSPVPIIPLRHVH
EDTVLGGYHVPAGTELAVNIYGCNMDKNVWENPEEWNPERFMKENETIDFQKTMAFGGGKRVCAGSLQALLTASIGI
GRMVQEFEWKLKDMTQEEVNTIGLTTQMLRPLRAIIKPRI (SEQ ID NO: 9)
>PpKO [Physcomitrella patens]
MAKHLATQLLQQWNEALKTMPPGFRTAGKILVWEELASNKVLITIALAWVLLFVARTCLRNKKRLPPAIPGGLPVLGNL
LQLTEKKPHRTFTAWSKEHGPIFTIKVGSVPQAVVNNSEIAKEVLVTKFASISKRQMPMALRVLTRDKTMVAMSDYGE
EHRMLKKLVMTNLLGPTTQNKNRSLRDDALIGMIEGVLAELKASPTSPKVVNVRDYVQRSLFPFALQQVFGYIPDQVE
VLELGTCVSTWDMFDALVVAPLSAVINVDWRDFFPALRWIPNRSVEDLVRTVDFKRNSIMKALIRAQRMRLANLKEPP
RCYADIALTEATHLTEKQLEMSLWEPIIESADTTLVTSEWAMYEIAKNPDCQDRLYREIVSVAGTERMVTEDDLPNMP
YLGAIIKETLRKYTPVPLIPSRFVEEDITLGGYDIPKGYQILVNLFAIANDPAVWSNPEKWDPERMLANKKVDMGFRDFS
LMPFGAGKRMCAGITQAMFIIPMNVAALVQHCEWRLSPQEISNINNKIEDVVYLTTHKLSPLSCEATPRISHRLP (SEQ
ID NO: 10)
>BmVO [Bacillus megaterium]
MTIKEMPQPKTFGELKNLPLLNTDKPVQALMKIADELGEIFKFEAPGRVTRYLSSQRLIKEACDESRFDKNLSQALKFV
RDFAGDGLATSWTHEKNWKKAHNILLPSFSQQAMKGYHAMMVDIAVQLVQKWERLNADEHIEVPEDMTRLTLDTIGL
CGFNYRFNSFYRDQPHPFITSMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEEAARVLVDPVPSYKQVKQLK
YVGMVLNEALRLWPTIPAFSLYAKEDTVLGGEYPLEKGDELMVLIPQLHRDKTIWGDDVEEFRPERFENPSAIPQHAF
KPFGNGQRACIGQQFALHEATLVLGMMLKHFDFEDHTNYELDIKETLTLKPEGFVVKAKSKKIPLGGIPSPSTEQSAKK
VRKKAENAHNTPLLVLYGSNMGTAEGTARDLADIAMSKGFAPQVATLDSHAGNLPREGAVLIVTASYNGHPPDNAKQ
FVDWLDQASADEVKGVRYSVFGCGDKNWATTYQKVPAFIDETLAAKGAENIADRGEADASDDFEGTYEEWREHMW
SDVAAYFNLDIENSEDNKSTLSLQFVDSAADMPLAKMHGAFSTNWASKELQQPGSARSTRHLEIELPKEASYQEGD
HLGVIPRNYEGIVNRVTARFGLDASQQIRLEAEEEKLAHLPLAKTVSVEELLQYVELQDPVIRTQLRAMAAKTVCPPH
KVELEALLEKQAYKEQVLAKRLTMLELLEKYPACEMKFSEFIALLPSIRPRYYSISSSPRVDEKQASITVSWSGEAWS
GYGEYKGIASNYLAELQEGDTITCFISTPQSEFTLPKDPETPLIMVGPGTGVAPFRGFVQARKQLKEQGQSLGEAHLY
FGCRSPHEDYLYQEELENAQSEGIITLHTAFSRMPNQPKTYVQHVMEQDGKKLIELLDQGAHFYICGDGSQMAPAVE
ATLMKSYADVHQVSEADARLWLQQLEEKGRYAKDVWAG (SEQ ID NO: 11)
>PsVO [Pleurotus sapidus]
MRYGCAAVALFYLTAMGKLHPLAIIPDYKGSMAASVTIFNKRTNPLDISVNQANDWPWRYAKTCVLSSDWALHEMIIH
LNNTHLVEEAVIVAAQRKLSPSHIVFRLLEPHWVVILSLNALARSVLIPEVIVPIAGFSAPHIFQFIRESFTNFDWKSLYV
PADLESRGFPVDQLNSPKFHNYAYARDINDMWTTLKKFVSSVLQDAQYYPDDASVAGDTQIQAWCDEMRSGMGAG
MTNFPESITTVDDLVNMVTMCIHIAAPQHTAVNYLQQYYQTFVSNKPSALFSPLPTSIAQLQKYTESDLMAALPLNAKR
QWLLMAQIPYLLSMQVQEDENIVTYAANASTDKDPIIASAGRQLAADLKKLAAVFLVNSAQLDDQNTPYDVLAPEQLA
NAIVI (SEQ ID NO: 12)
>PoLO [Pleurotus ostreatus]
MAPTMSLSRSALKNVHLPYMVQHPEPTDCSTAMKHAAEGYDRARQMIAFLFDILDYESSVPQKFTPEEKKEKYTWSH
SDKFPPHLAIIPEDIDVPAYIIFSIVRLVQTLSIMSGIQCNERLAPGPEQNTMEKLTKWNAERHKNQGWVKDMFNEPNIG
LRNDWYTDAVFAQQFFTGPNPTTITLASDTWMKAFTEEAASQGKRDLISLFRSAPPNSFYVQDFSDFRARMGAKPDE
ELCATSDGGVTRYGCAAVALFYLPPTGELHPLAIVPDYKGSMAASITLFNKRVDPSDASVDQANDWPWRYAKTCVLS
ADWVLHEMIIHLNNTHLVQEAVIVAVQRTLPDSHIVFRLLKPHWVVTLSLNAQARSVLIPEVIVPIAGFSELRIFQFVGHA
FTNFDWKALYVPTDLEFRGFPLDRLDDDKFHNYAYAKDIKDMWMALRKFVSSVLKDGKYYPDDSAVAADAQIQDWC
DEMRSEKGAGMKKFPESISTLDDLIDMVTMCIHIAAPQHTAVNYLQQYYQTFVPNKPSALFSPLPTLLSQLESYTESDL
MAALPLGAKQEWLLMAQVPYLLSKEVEQDGNIVTYAGTASNNEDPIIAAAGKELSADLVILAGVFLKNSEKLDDQNTAY
NVLAPDQLANAIVI (SEQ ID NO: 13)
>CiVO [Cichorium intybus]
MEISIPTTLGLAVIIFIIFKLLTRTTSKKNLLPEPWRLPIIGHMHHLIGTMPHRGVMELARKHGSLMHLQLGEVSTIWSSP
RWAKEVLTTYDITFANRPETLTGEIVAYHNTDIVLAPYGEYWRQLRKLCTLELLSNKKVKSFQSLREEECWNLVKDIRS
TGQGSPINLSENIFKMIATILSRAAFGKGIKDQMKFTELVKEILRLTGGFDVADIFPSKKLLHHLSGKRAKLTNIHNKLDN
LINNIIAEHPGNRTSSSQETLLDVLLRLKESAEFPLTADNVKAVILDMFGAGTDTSSATIEWAISELIRCPRAMEKVQTEL
RQALNGKERIQEEDLQELNYLKLVIKETLRLHPPLPLVMPRECREPCVLGGYDIPSKTKLIVNVFAINRDPEYWKDAET
FMPERFENSPITVMGSEYEYLPFGAGRRMCPGAALGLANVELPLAHILYFNWKLPNGKTFEDLDMTESFGATVQRKT
ELLLVPTDFQTLTAST (SEQ ID NO: 14)
>HaGAO [Helianthus annuus]
MEVSLTTSIALATIVFFLYKLLTRPTSSKNRLPEPWRLPIIGHMHHLIGTMPHRGVMDLARKYGSLMHLQLGEVSAIWS
SPKWAKEILTTYDIPFANRPETLIGEIIAYHNTDIVLAPYGEYWRQLRKLCTLELLSVKKVKSFQSLREEECWNLVQEIK
ASGSGTPFNLSEGIFKVIATVLSRAAFGKGIKDQKQFTEIVKEILRETGGFDVADIFPSKKFLHHLSGKRGRLTSIHNKLD
SLINNLVAEHTVSKSSKVNETLLDVLLRLKNSEEFPLTADNVKAIILDMFGAGTDTSSATVEWAISELIRCPRAMEKVQA
ELRQALNGKERIKEEEIQDLPYLNLVIRETLRLHPPLPLVMPRECRQAMNLAGYDVANKTKLIVNVFAINRDPEYWKDA
ESFNPERFENSNTTIMGADYEYLPFGAGRRMCPGSALGLANVQLPLANILYYFKWKLPNGASHDQLDMTESFGATVQ
RKTELMLVPSF (SEQ ID NO: 15)
CYTOCHROME P450 REDUCTASE PARTNERS
>SrCPR [Stevia rebaudiana]
MQSDSVKVSPFDLVSAAMNGKAMEKLNASESEDPTTLPALKMLVENRELLTLFTTSFAVLIGCLVFLMWRRSSSKKLV
QDPVPQVIWKKKEKESEVDDGKKKVSIFYGTQTGTAEGFAKALVEEAKVRYEKTSFKVIDLDDYAADDDEYEEKLKK
ESLAFFFLATYGDGEPTDNAANFYKWFTEGDDKGEWLKKLQYGVFGLGNRQYEHFNKIAIWDDKLTEMGAKRLVPV
GLGDDDQCIEDDFTAWKELVWPELDQLLRDEDDTSVTTPYTAAVLEYRWYHDKPADSYAEDQTHINGHWHDAQH
PSRSNVAFKKELHTSQSDRSCTHLEFDISHTGLSYETGDHVGVYSENLSEWDEALKLLGLSPDTYFSVHADKEDGTP
IGGASLPPPFPPCTLRDALTRYADVLSSPKKVALLALAAHASDPSEADRLKFLASPAGKDEYAQWIVANQRSLLEVMQ
SFPSAKPPLGVFFAAVAPRLQPRYYSISSSPKMSPNRIHVTCALVYETTPAGRIHRGLCSTWMKNAVPLTESPDCSQA
SIFVRTSNFRLPVDPKVPVIMIGPGTGLAPFRGFLQERLALKESGTELGSSIFFFGCRNRKVDFIYEDELNNFVETGALS
ELIVAFSREGTAKEYVQHKMSQKASDIWKLLSEGAYLYVCGDAKGMAKDVHRTLHTIVQEQGSLDSSKAELYVKNLQ
MSGRYLRDVW (SEQ ID NO: 33)
>AtCPR [Arabidopsis thaliana]
MTSALYASDLFKQLKSIMGTDSLSDDWLVIATTSLALVAGFWLLWKKTTADRSGELKPLMIPKSLMAKDEDDDLDLG
SGKTRVSIFFGTQTGTAEGFAKALSEEIKARYEKAAVKVIDLDDYAADDDQYEEKLKKETLAFFCVATYGDGEPTDNA
ARFSKWFTEENERDIKLQQLAYGVFALGNRQYEHFNKIGIVLDEELCKKGAKRLIEVGLGDDDQSIEDDFNAWKESLW
SELDKLLKDEDDKSVATPYTAVIPEYRWTHDPRFTTQKSMESNVANGNTTIDIHHPCRVDVAVQKELHTHESDRSCI
HLEFDISRTGITYETGDHVGVYAENHVEIVEEAGKLLGHSLDLVFSIHADKEDGSPLESAVPPPFPGPCTLGTGLARYA
DLLNPPRKSALVALAAYATEPSEAEKLKHLTSPDGKDEYSQWIVASQRSLLEVMAAFPSAKPPLGVFFAAIAPRLQPR
YYSISSCQDWAPSRVHVTSALVYGPTPTGRIHKGVCSTWMKNAVPAEKSHECSGAPIFIRASNFKLPSNPSTPIVMVG
PGTGLAPFRGFLQERMALKEDGEELGSSLLFFGCRNRQMDFIYEDELNNFVDQGVISELIMAFSREGAQKEYVQHKM
MEKAAQVWDLIKEEGYLYVCGDAKGMARDVHRTLHTIVQEQEGVSSSEAEAIVKKLQTEGRYLRDVW (SEQ ID NO:
34)
>TcCPR [Taxus cuspidata]
MQANSNTVEGASQGKSLLDISRLDHIFALLLNGKGGDLGAMTGSALILTENSQNLMILTTALAVLVACVFFFVWRRGG
SDTQKPAVRPTPLVKEEDEEEEDDSAKKKVTIFFGTQTGTAEGFAKALAEEAKARYEKAVFMNDLDNYAADDEQYE
EKLKKEKLAFFMLATYGDGEPTDNAARFYKWFLEGKEREPWLSDLTYGVFGLGNRQYEHFNKVAKAVDEVLIEQGA
KRLVPVGLGDDDQCIEDDFTAWREQVWPELDQLLRDEDDEPTSATPYTAAIPEYRVEIYDSWSVYEETHALKQNGQ
AVYDIHHPCRSNVAVRRELHTPLSDRSCIHLEFDISDTGLIYETGDHVGVHTENSIETVEEAAKLLGYQLDTIFSVHGDK
EDGTPLGGSSLPPPFPGPCTLRTALARYADLLNPPRKAAFLALAAHASDPAEAERLKFLSSPAGKDEYSQWVTASQR
SLLEIMAEFPSAKPPLGVFFAAIAPRLQPRYYSISSSPRFAPSRIHVTCALVYGPSPTGRIHKGVCSNWMKNSLPSEET
HDCSWAPVFVRQSNFKLPADSTTPIVMVGPGTGFAPFRGFLQERAKLQEAGEKLGPAVLFFGCRNRQMDYIYEDEL
KGYVEKGILTNLIVAFSREGATKEYVQHKMLEKASDTWSLIAQGGYLYVCGDAKGMARDVHRTLHTIVQEQESVDSS
KAEFLVKKLQMDGRYLRDIW (SEQ ID NO: 35)
>AaCPR [Artemisia annua]
MAQSTTSVKLSPFDLMTALLNGKVSFDTSNTSDINIPLAVFMENRELLMILTTSVAVLIGCWVLVWRRSSSAAKKAAE
SPVIVVPKKVTEDEVDDGRKKVTVFFGTQTGTAEGFAKALVEEAKARYEKAVFKVIDLDDYAAEDDEYEEKLKKESLA
FFFLATYGDGEPTDNAARFYKWFTEGEEKGEWLDKLQYAVFGLGNRQYEHFNKIAKVVDEKLVEQGAKRLVPVGMG
DDDQCIEDDFTAWKELVWPELDQLLRDEDDTSVATPYTAAVAEYRVVFHDKPETYDQDQLTNGHAVHDAQHPCRSN
VAVKKELHSPLSDRSCTHLEFDISNTGLSYETGDHVGVYVENLSEVVDEAEKLIGLPPHTYFSVHADNEDGTPLGGAS
LPPPFPPCTLRKALASYADVLSSPKKSALLALAAHATDSTEADRLKFLASPAGKDEYAQWIVASHRSLLEVMEAFPSA
KPPLGVFFASVAPRLQPRYYSISSSPRFAPNRIHVTCALVYEQTPSGRVHKGVCSTWMKNAVPMTESQDCSWAPIYV
RTSNFRLPSDPKVPVIMIGPGTGLAPFRGFLQERLAQKEAGTELGTAILFFGCRNRKVDFIYEDELNNFVETGALSELV
TAFSREGATKEYVQHKMTQKASDIWNLLSEGAYLYVCGDAKGMAKDVHRTLHTIVQEQGSLDSSKAELYVKNLQMA
GRYLRDVW (SEQ ID NO: 36)
>AtCPR1 [Arabidopsis thaliana]
MATSALYASDLFKQLKSIMGTDSLSDDVVLVIATTSLALVAGFWLLWKKTTADRSGELKPLMIPKSLMAKDEDDDLDL
GSGKTRVSIFFGTQTGTAEGFAKALSEEIKARYEKAAVKVIDLDDYAADDDQYEEKLKKETLAFFCVATYGDGEPTDN
AARFYKWFTEENERDIKLQQLAYGVFALGNRQYEHFNKIGIVLDEELCKKGAKRLIEVGLGDDDQSIEDDFNAWKESL
WSELDKLLKDEDDKSVATPYTAVIPEYRWTHDPRFTTQKSMESNVANGNITTIDIHHPCRVDVAVQKELHTHESDRS
CIHLEFDISRTGITYETGDHVGVYAENHVEIVEEAGKLLGHSLDLVFSIHADKEDGSPLESAVPPPFPGPCTLGTGLAR
YADLLNPPRKSALVALAAYATEPSEAEKLKHLTSPDGKDEYSQWIVASQRSLLEVMAAFPSAKPPLGVFFAAIAPRLQ
PRYYSISSSPRLAPSRVHVTSALVYGPTPTGRIHKGVCSTWMKNAVPAEKSHECSGAPIFIRASNFKLPSNPSTPIVMV
GPGTGLAPFRGFLQERMALKEDGEELGSSLLFFGCRNRQMDFIYEDELNNFVDQGVISELIMAFSREGAQKEYVQHK
MMEKAAQVWDLIKEEGYLYVCGDAKGMARDVHRTLHTIVQEQEGVSSSEAEAIVKKLQTEGRYLRDVW (SEQ ID
NO: 37)
>AtCPR2 [Arabidopsis thaliana]
MASSSSSSSTSMIDLMAAIIKGEPVIVSDPANASAYESVAAELSSMLIENRQFAMIVITSIAVLIGCIVMLVWRRSGSGN
SKRVEPLKPLVIKPREEEIDDGRKKVTIFFGTQTGTAEGFAKALGEEAKARYEKTRFKIVDLDDYAADDDEYEEKLKKE
DVAFFFLATYGDGEPTDNAARFYKWFTEGNDRGEWLKNLKYGVFGLGNRQYEHFNKVAKVVDDILVEQGAQRLVQV
GLGDDDQCIEDDFTAWREALWPELDTILREEGDTAVATPYTAAVLEYRVSIHDSEDAKFNDINMANGNGYTVFDAQH
PYKANVAVKRELHTPESDRSCIHLEFDIAGSGLTYETGDHVGVLCDNLSETVDEALRLLDMSPDTYFSLHAEKEDGTPI
SSSLPPPFPPCNLRTALTRYACLLSSPKKSALVALAAHASDPTEAERLKHLASPAGKDEYSKWVVESQRSLLEVMAEF
PSAKPPLGVFFAGVAPRLQPRFYSISSSPKIAETRIHVTCALVYEKMPTGRIHKGVCSTWMKNAVPYEKSENCSSAPIF
VRQSNFKLPSDSKVPIIMIGPGTGLAPFRGFLQERLALVESGVELGPSVLFFGCRNRRMDFIYEEELQRFVESGALAEL
SVAFSREGPTKEYVQHKMMDKASDIWNMISQGAYLYVCGDAKGMARDVHRSLHTIAQEQGSMDSTKAEGFVKNLQ
TSGRYLRDVW (SEQ ID NO: 38)
>ATR2 [Arabidopsis thaliana]
MASSSSSSSTSMIDLMAAIIKGEPVIVSDPANASAYESVAAELSSMLIENRQFAMIVITSIAVLIGCIVMLVWRRSGSGN
SKRVEPLKPLVIKPREEEIDDGRKKVTIFFGTQTGTAEGFAKALGEEAKARYEKTRFKIVDLDDYAADDDEYEEKLKKE
DVAFFFLATYGDGEPTDNAARFYKWFTEGNDRGEWLKNLKYGVFGLGNRQYEHFNKVAKVVDDILVEQGAQRLVQV
GLGDDDQCIEDDFTAWREALWPELDTILREEGDTAVATPYTAAVLEYRVSIHDSEDAKFNDITLANGNGYTVFDAQHP
YKANVAVKRELHTPESDRSCIHLEFDIAGSGLIMKLGDHVGVLCDNLSETVDEALRLLDMSPDTYFSLHAEKEDGTPI
SSSLPPPFPPCNLRTALTRYACLLSSPKKSALVALAAHASDPTEAERLKHLASPAGKDEYSKWVVESQRSLLEVMAEF
PSAKPPLGVFFAGVAPRLQPRFYSISSSPKIAETRIHVTCALVYEKMPTGRIHKGVCSTWMKNAVPYEKSEKLFLGRPI
FVRQSNFKLPSDSKVPIIMIGPGTGLAPFRGFLQERLALVESGVELGPSVLFFGCRNRRMDFIYEEELQRFVESGALAE
LSVAFSREGPTKEYVQHKMMDKASDIWNMISQGAYLYVCGDAKGMARDVHRSLHTIAQEQGSMDSTKAEGFVKNL
QTSGRYLRDVW (SEQ ID NO: 39)
>SrCPR1 [Stevia rebaudiana]
MAQSDSVKVSPFDLVSAAMNGKAMEKLNASESEDPTTLPALKMLVENRELLTLFTTSFAVLIGCLVFLMWRRSSSKKL
VQDPVPQVIVVKKKEKESEVDDGKKKVSIFYGTQTGTAEGFAKALVEEAKVRYEKTSFKVIDLDDYAADDDEYEEKLK
KESLAFFFLATYGDGEPTDNAANFYKWFTEGDDKGELLKKLQYGVFGLGNRQYEHFNKIAIVVDDKLTEMGAKRLVP
VGLGDDDQCIEDDFTAWKELVWPELDQLLRDEDDTSVTTPYTAAVLEYRWYHDKPADSYAEDQTHTNIGHWHDAQ
HPSRSNVAFKKELHTSQSDRSCTHLEFDISHTGLSYETGDHVGVYSENLSEVVDEALKLLGLSPDTYFSVHADKEDG
TPIGGASLPPPFPPCTLRDALTRYADVLSSPKKVALLALAAHASDPSEADRLKFLASPAGKDEYAQWIVANQRSLLEV
MQSFPSAKPPLGVFFAAVAPRLQPRYYSISSSPKMSPNRIHVTCALVYETTPAGRIHRGLCSTWMKNAVPLTESPDC
SQASIFVRTSNFRLPVDPKVPVIMIGPGTGLAPFRGFLQERLALKESGTELGSSIFFFGCRNRKVDFIYEDELNNFVET
GALSELIVAFSREGTAKEYVQHKMSQKASDIWKLLSEGAYLYVCGDAKGMAKDVHRTLHTIVQEQGSLDSSKAELYV
KNLQMSGRYLRDVW (SEQ ID NO: 40)
>SrCPR2 [Stevia rebaudiana]
MAQSESVEASTIDLMTAVLKDTVIDTANASDNGDSKMPPALAMMFEIRDLLLILTTSVAVLVGCFVVLVWKRSSGKKS
GKELEPPKIVVPKRRLEQEVDDGKKKVTIFFGTQTGTAEGFAKALFEEAKARYEKAAFKVIDLDDYAADLDEYAEKLKK
ETYAFFFLATYGDGEPTDNAAKFYKWFTEGDEKGVWLQKLQYGVFGLGNRQYEHFNKIGIVVDDGLTEQGAKRIVPV
GLGDDDQSIEDDFSAWKELVWPELDLLLRDEDDKAAATPYTAAIPEYRVVFHDKPDAFSDDHTQINGHAVHDAQHP
CRSNVAVKKELHTPESDRSCTHLEFDISHTGLSYETGDHVGVYCENLIEVVEEAGKLLGLSTDTYFSLHIDNEDGSPL
GGPSLQPPFPPCTLRKALTNYADLLSSPKKSTLLALAAHASDPTEADRLRFLASREGKDEYAEWVVANQRSLLEVME
AFPSARPPLGVFFAAVAPRLQPRYYSISSSPKMEPNRIHVTCALVYEKTPAGRIHKGICSTWMKNAVPLTESQDCSWA
PIFVRTSNFRLPIDPKVPVIMIGPGTGLAPFRGFLQERLALKESGTELGSSILFFGCRNRKVDYIYENELNNFVENGALS
ELDVAFSRDGPTKEYVQHKMTQKASEIWNMLSEGAYLYVCGDAKGMAKDVHRTLHTIVQEQGSLDSSKAELYVKNL
QMSGRYLRDVW (SEQ ID NO: 41)
>SrCPR3 [Stevia rebaudiana]
MAQSNSVKISPLDLVTALFSGKVLDTSNASESGESAMLPTIAMIMENRELLMILTTSVAVLIGCWVLVWRRSSIKKSAL
EPPVIVVPKRVQEEEVDDGKKKVTVFFGTQTGTAEGFAKALVEEAKARYEKAVFKVIDLDDYAADDDEYEEKLKKESL
AFFFLATYGDGEPTDNAARFYKWFTEGDAKGEWLNKLQYGVFGLGNRQYEHFNKIAKVVDDGLVEQGAKRLVPVGL
GDDDQCIEDDFTAWKELVWPELDQLLRDEDDTTVATPYTAAVAEYRVVFHEKPDALSEDYSYTNGHAVHDAQHPCR
SNVAVKKELHSPESDRSCTHLEFDISNTGLSYETGDHVGVYCENLSEWNDAERLVGLPPDTYFSIHTDSEDGSPLG
GASLPPPFPPCTLRKALTCYADVLSSPKKSALLALAAHATDPSEADRLKFLASPAGKDEYSQWIVASQRSLLEVMEAF
PSAKPSLGVFFASVAPRLQPRYYSISSSPKMAPDRIHVTCALVYEKTPAGRIHKGVCSTWMKNAVPMTESQDCSWAP
IYVRTSNFRLPSDPKVPVIMIGPGTGLAPFRGFLQERLALKEAGTDLGLSILFFGCRNRKVDFIYENELNNFVETGALSE
LIVAFSREGPTKEYVQHKMSEKASDIWNLLSEGAYLYVCGDAKGMAKDVHRTLHTIVQEQGSLDSSKAELYVKNLQM
SGRYLRDVW (SEQ ID NO: 42)
>PgCPR [Pelargonium graveolens]
MAQSSSGSMSPFDFMTAIIKGKMEPSNASLGAAGEVTAMILDNRELVMILTTSIAVLIGCWVFIWRRSSSQTPTAVQP
LKPLLAKETESEVDDGKQKVTIFFGTQTGTAEGFAKALADEAKARYDKVTFKVVDLDDYAADDEEYEEKLKKETLAFF
FLATYGDGEPTDNAARFYKWFLEGKERGEWLQNLKFGVFGLGNRQYEHFNKIAIVVDEILAEQGGKRLISVGLGDDD
QCIEDDFTAWRESLWPELDQLLRDEDDTTVSTPYTAAVLEYRVVFHDPADAPTLEKSYSNANGHSVVDAQHPLRANV
AVRRELHTPASDRSCTHLEFDISGTGIAYETGDHVGVYCENLAETVEEALELLGLSPDTYFSVHADKEDGTPLSGSSL
PPPFPPCTLRTALTLHADLLSSPKKSALLALAAHASDPTEADRLRHLASPAGKDEYAQWIVASQRSLLEVMAEFPSAK
PPLGVFFASVAPRLQPRYYSISSSPRIAPSRIHVTCALVYEKTPTGRVHKGVCSTWMKNSVPSEKSDECSWAPIFVRQ
SNFKLPADAKVPIIMIGPGTGLAPFRGFLQERLALKEAGTELGPSILFFGCRNSKMDYIYEDELDNFVQNGALSELVLAF
SREGPTKEYVQHKMMEKASDIWNLISQGAYLYVCGDAKGMARDVHRTLHTIAQEQGSLDSSKAESMVKNLQMSGR
YLRDVW (SEQ ID NO: 43)
LINKER SEQUENCES
GSGGGGS (SEQ ID NO: 44)
GSGEAAAK (SEQ ID NO: 45)
GSGEAAAKEAAAK (SEQ ID NO: 46)
GSGMGSSSN (SEQ ID NO: 47).
P450 ENZYMES WITH TRANSMEMBRANE DOMAIN DELETED
>t22ZzHO [Zingiber zerumbet]
LLIKRSSRSSVHKQQVLLASLPPSPPRLPLIGNIHQLVGGNPHRILLQLARTHGPLICLRLGQVDQVVASSVEAVEEIIKR
HDLKFADRPRDLIFSRIFFYDGNAVVMTPYGGEWKQMRKIYAMELLNSRRVKSFAAIREDVARKLTGEIAHKAFAQTP
VINLSEMVMSMINAIVIRVAFGDKCKQQAYFLHLVKEAMSYVSSFSVADMYPSLKFLDTLTGLKSKLEGVHGKLDKVFD
EIIAQRQAALAAEQAEEDLIIDVLLKLKDEGNQEFPITYTSVKAIVMEIFLAGTETSSSVIDWVMSELIKNPKAMEKVQKE
MREAMQGKTKLEESDIPKFSYLNLVIKETLRLHPPGPLLFPRECRETCEVMGYRVPAGARLLINAFALSRDEKYWGSD
AESFKPERFEGISVDFKGSNFEFMPFGAGRRICPGMTFGISSVEVALAHLLFHFDWQLPQGMKIEDLDMMEVSGMSA
TRRSPLLVLAKLIIPLP (SEQ ID NO: 48)
>t20BsGAO [Barnadesia spinosa]
LLTGSKSTKNSLPEAWRLPIIGHMHHLVGTLPHRGVTDMARKYGSLMHLQLGEVSTIVVSSPRWAKEVLTTYDITFAN
RPETLTGEIVAYHNTDIVLSPYGEYWRQLRKLCTLELLSAKKVKSFQSLREEECWNLVKEVRSSGSGSPVDLSESIFKL
IATILSRAAFGKGIKDQREFTEIVKEILRLTGGFDVADIFPSKKILHHLSGKRAKLTNIHNKLDSLINNIVSEHPGSRTSSS
QESLLDVLLRLKDSAELPLTSDNVKAVILDMFGAGTDTSSATIEWAISELIRCPRAMEKVQTELRQALNGKERIQEEDIQ
ELSYLKLVIKETLRLHPPLPLVMPRECREPCVLAGYEIPTKTKLIVNVFAINRDPEYWKDAETFMPERFENSPINIMGSE
YEYLPFGAGRRMCPGAALGLANVELPLAHILYYFNWKLPNGARLDELDMSECFGATVQRKSELLLVPTAYKTANNSA
(SEQ ID NO: 49)
>t16HmPO [Hyoscyamus muticus]
FLLRKWKNSNSQSKKLPPGPWKLPLLGSMLHMVGGLPHHVLRDLAKKYGPLMHLQLGEVSAVVVISPDMAKEVLKT
HDIAFASRPKLLAPEIVCYNRSDIAFCPYGDYWRQMRKICVLEVLSAKNVRSFSSIRRDEVLRLVNFVRSSTSEPVNFT
ERLFLFTSSMTCRSAFGKVFKEQETFIQLIKEVIGLAGGFDVADIFPSLKFLHVLTGMEGKIMKAHHKVDAIVEDVINEH
KKNLAMGKTNGALGGEDLIDVLLRLMNDGGLQFPITNDNIKAIIFDMFAAGTETSSSTLVWAMVQMMRNPTILAKAQA
EVREAFKGKETFDENDVEELKYLKLVIKETLRLHPPVPLLVPRECREETEINGYTIPVKTKVMVNVWALGRDPKYWDD
ADNFKPERFEQCSVDFIGNNFEYLPFGGGRRICPGISFGLANVYLPLAQLLYHFDWKLPTGMEPKDLDLTELVGVTAA
RKSDLMLVATPYQPSRE (SEQ ID NO: 50)
>t19LsGAO [Lactuca sativa]
KLATRPKSTKKQLPEASRLPIIGHMHHLIGTMPHRGVMDLARKHGSLMHLQLGEVSTIVVSSPKWAKEILTTYDITFAN
RPETLIGEIIAYHNTDIVLAPYGEYWRQLRKLCTLELLSVKKVKSFQSIREEECWNLVKEVKESGSGKPINLSESIFTMI
ATILSRAAFGKGIKDQREFTEIVKEILRQTGGFDVADIFPSKKFLHHLSGKRARLTSIHKKLDNLINNIVAEHHVSTSSKA
NETLLDVLLRLKDSAEFPLTADNVKAIILDMFGAGTDTSSATVEWAISELIRCPRAMEKVQAELRQALNGKEKIQEEDIQ
DLAYLNLVIRETLRLHPPLPLVMPRECREPVNLAGYEIANKTKLIVNVFAINRDPEYWKDAEAFIPERFENNPNNIMGAD
YEYLPFGAGRRMCPGAALGLANVQLPLANILYHFNWKLPNGASHDQLDMTESFGATVQRKTELLLVPSF (SEQ ID
NO: 51)
>t16NtEAO [Nicotiani tabacum]
FLLRKWKNSNSQSKKLPPGPWKIPILGSMLHMIGGEPHHVLRDLAKKYGPLMHLQLGEISAVVVISRDMAKEVLKTH
DVVFASRPKIVAMDIICYNQSDIAFSPYGDHWRQMRKICVMELLNAKNVRSFSSIRRDEVVRLIDSIRSDSSSGELVNF
TQRIIWFASSMTCRSAFGQVLKGQDIFAKKIREVIGLAEGFDVVDIFPTYKFLHVLSGMKRKLLNAHLKVDAIVEDVINE
HKKNLAAGKSNGALGGEDLIDVLLRLMNDTSLQFPITNDNIKAVIVDMFAAGTETSSTTTVWAMAEMMKNPSVFTKAQ
AEVREAFRDKVSFDENDVEELKYLKLVIKETLRLHPPSPLLVPRECREDTDINGYTIPAKTKVMVNVWALGRDPKYWD
DAESFKPERFEQCSVDFFGNNFEFLPFGGGRRICPGMSFGLANLYLPLAQLLYHFDWKLPTGIMPRDLDLTELSGITIA
RKGGLYLNATPYQPSRE (SEQ ID NO: 52)
>t26CpVO [Citrus x paradisi]
WVWLRPKKLEKFLRQQGLKGNSYRLLFGDLKENSIELKEAKARPLSLDDDIAIRVNPFLHKLVNDYGKNSFMWFGPTP
RVNIMNPDQIKAIFTKINDFQKVNSIPLARLLIVGLATLEGEKWAKHRKLINPAFHQEKLKLMLPAFYLSCIEIITKWEKQM
SVEGSSELDVWPYLANLTSDVISRTAFGSSYEEGRRIFQLQAELAELTMQVFRSVHIPGWRFLPTKRNRRMKEIDKEI
RASLMGIIKNREKAMRAGEAANNDLLGILMETSFREIEEHGNNKNVGFSMNDVIEECKLFYFAGQETTSVLLNWTMVL
LSKHQDWQERARQEVLQVFGNNKPDYDGLNHLKIVQMILYEVLRLYPPVTVLSRAVFKETKLGNLTLPAGVQIGLPMI
LVHQDPELWGDDAVEFKPERFAEGISKAAKNQVSYFPFALGPRICVGQNFALVEAKMATAMILQNYSFELSPSYVHA
PTAVPILHPELGTQLILRKLWCKNN (SEQ ID NO: 53)
>t23AaAO [Artemesia annua]
FVYKFATRSKSTKKSLPEPWRLPIIGHMHHLIGTTPHRGVRDLARKYGSLMHLQLGEVPTIVVSSPKWAKEILTTYDITF
ANRPETLTGEIVLYHNTDWLAPYGEYWRQLRKICTLELLSVKKVKSFQSLREEECWNLVQEIKASGSGRPVNLSENV
FKLIATILSRAAFGKGIKDQKELTEIVKEILRQTGGFDVADIFPSKKFLHHLSGKRARLTSLRKKIDNLIDNLVAEHTVNTS
SKTNETLLDVLLRLKDSAEFPLTSDNIKAIILDMFGAGTDTSSSTIEWAISELIKCPKAMEKVQAELRKALNGKEKIHEEDI
QELSYLNMVIKETLRLHPPLPLVLPRECRQPVNLAGYNIPNKTKLIVNVFAINRDPEYWKDAEAFIPERFENSSATVMG
AEYEYLPFGAGRRMCPGAALGLANVQLPLANILYHFNWKLPNGVSYDQIDMTESSGATMQRKTELLLVPSF (SEQ ID
NO: 54)
>t21AtKO [Arabidopsis thaliana]
FFFKKLLSFSRKNMSEVSTLPSVPVVPGFPVIGNLLQLKEKKPHKTFTRWSEIYGPIYSIKMGSSSLIVLNSTETAKEAM
VTRFSSISTRKLSNALTVLTCDKSMVATSDYDDFHKLVKRCLLNGLLGANAQKRKRHYRDALIENVSSKLHAHARDHP
QEPVNFRAIFEHELFGVALKQAFGKDVESIYVKELGVTLSKDEIFKVLVHDMMEGAIDVDWRDFFPYLKWIPNKSFEAR
IQQKHKRRLAVMNALIQDRLKQNGSESDDDCYLNFLMSEAKTLIKEQIAILVWETIIETADTTLVTTEWAIYELAKHPSV
QDRLCKEIQNVCGGEKFKEEQLSQVPYLNGVFHETLRKYSPAPLVPIRYAHEDTQIGGYHVPAGSEIAINIYGCNMDK
KRWERPEDWWPERFLDDGKYETSDLHKTMAFGAGKRVCAGALQASLMAGIAIGRLVQEFEWKLRDGEEENVDTYG
LTSQKLYPLMAIINPRRS (SEQ ID NO: 55)
>t30SrKO [Stevia rebaudiana]
WYLKSYTSARRSQSNHLPRVPEVPGVPLLGNLLQLKEKKPYMTFTRWAATYGPIYSIKTGATSMVVVSSNEIAKEALV
TRFQSISTRNLSKALKVLTADKTMVAMSDYDDYHKTVKRHILTAVLGPNAQKKHRIHRDIMMDNISTQLHEFVKNNPE
QEEVDLRKIFQSELFGLAMRQALGKDVESLYVEDLKITMNRDEIFQVLVVDPMMGAIDVDWRDFFPYLKWVPNKKFE
NTIQQMYIRREAVMKSLIKEHKKRIASGEKLNSYIDYLLSEAQTLTDQQLLMSLWEPIIESSDTTMVTTEWAMYELAKNP
KLQDRLYRDIKSVCGSEKITEEHLSQLPYITAIFHETLRRHSPVPIIPLRHVHEDTVLGGYHVPAGTELAVNIYGCNMDK
NVWENPEEWNPERFMKENETIDFQKTMAFGGGKRVCAGSLQALLTASIGIGRMVQEFEWKLKDMTQEEVNTIGLTT
QMLRPLRAIIKPRI (SEQ ID NO: 56)
>t52PpKO [Physcomitrella patens]
FVARTCLRNKKRLPPAIPGGLPVLGNLLQLTEKKPHRTFTAWSKEHGPIFTIKVGSVPQAVVNNSEIAKEVLVTKFASI
SKRQMPMALRVLTRDKTMVAMSDYGEEHRMLKKLVMTNLLGPTTQNKNRSLRDDALIGMIEGVLAELKASPTSPKV
VNVRDYVQRSLFPFALQQVFGYIPDQVEVLELGTCVSTWDMFDALVVAPLSAVINVDWRDFFPALRWIPNRSVEDLV
RTVDFKRNSIMKALIRAQRMRLANLKEPPRCYADIALTEATHLTEKQLEMSLWEPIIESADTTLVTSEWAMYEIAKNPD
CQDRLYREIVSVAGTERMVTEDDLPNMPYLGAIIKETLRKYTPVPLIPSRFVEEDITLGGYDIPKGYQILVNLFAIANDPA
VWSNPEKWDPERMLANKKVDMGFRDFSLMPFGAGKRMCAGITQAMFIIPMNVAALVQHCEWRLSPQEISNINNKIE
DVVYLTTHKLSPLSCEATPRISHRLP (SEQ ID NO: 57)
>t15PsVO [Pleurotus sapidus]
MGKLHPLAIIPDYKGSMAASVTIFNKRTNPLDISVNQANDWPWRYAKTCVLSSDWALHEMIIHLNNTHLVEEAVIVAAQ
RKLSPSHIVFRLLEPHWVVILSLNALARSVLIPEVIVPIAGFSAPHIFQFIRESFINFDWKSLYVPADLESRGFPVDQLNS
PKFHNYAYARDINDMWTTLKKFVSSVLQDAQYYPDDASVAGDTQIQAWCDEMRSGMGAGMTNFPESITTVDDLVN
MVTMCIHIAAPQHTAVNYLQQYYQTFVSNKPSALFSPLPTSIAQLQKYTESDLMAALPLNAKRQWLLMAQIPYLLSMQ
VQEDENIVTYAANASTDKDPIIASAGRQLAADLKKLAAVFLVNSAQLDDQNTPYDVLAPEQLANAIVI (SEQ ID NO: 58)
>t20CiVO [Cichorium intybus]
LLTRTTSKKNLLPEPWRLPIIGHMHHLIGTMPHRGVMELARKHGSLMHLQLGEVSTIVVSSPRWAKEVLTTYDITFANR
PETLTGEIVAYHNTDIVLAPYGEYWRQLRKLCTLELLSNKKVKSFQSLREEECWNLVKDIRSTGQGSPINLSENIFKMIA
TILSRAAFGKGIKDQMKFTELVKEILRLTGGFDVADIFPSKKLLHHLSGKRAKLTNIHNKLDNLINNIIAEHPGNRTSSSQ
ETLLDVLLRLKESAEFPLTADNVKAVILDMFGAGTDTSSATIEWAISELIRCPRAMEKVQTELRQALNGKERIQEEDLQE
LNYLKLVIKETLRLHPPLPLVMPRECREPCVLGGYDIPSKTKLIVNVFAINRDPEYWKDAETFMPERFENSPITVMGSE
YEYLPFGAGRRMCPGAALGLANVELPLAHILYFNWKLPNGKTFEDLDMTESFGATVQRKTELLLVPIDFQTLTAST
(SEQ ID NO: 59)
>t20HaGAO [Helianthus annuus]
LLTRPTSSKNRLPEPWRLPIIGHMHHLIGTMPHRGVMDLARKYGSLMHLQLGEVSAIVVSSPKWAKEILTTYDIPFANR
PETLIGEIIAYHNTDIVLAPYGEYWRQLRKLCTLELLSVKKVKSFQSLREEECWNLVQEIKASGSGTPFNLSEGIFKVIA
TVLSRAAFGKGIKDQKQFTEIVKEILRETGGFDVADIFPSKKFLHHLSGKRGRLTSIHNKLDSLINNLVAEHTVSKSSKV
NETLLDVLLRLKNSEEFPLTADNVKAIILDMFGAGTDTSSATVEWAISELIRCPRAMEKVQAELRQALNGKERIKEEEIQ
DLPYLNLVIRETLRLHPPLPLVMPRECRQAMNLAGYDVANKTKLIVNVFAINRDPEYWKDAESFNPERFENSNTTIMG
ADYEYLPFGAGRRMCPGSALGLANVQLPLANILYYFKWKLPNGASHDQLDMTESFGATVQRKTELMLVPSF (SEQ ID
NO: 60)
Bacillus subtilis TRANSMEMBRANE PROTEINS
>ATPL_BACSU [Bacillus subtilis]
MNLIAAAIAIGLGALGAGIGNGLIVSRTVEGIARQPEAGKELRILMFMGIALVEALPHAVVIAFLAFFG (SEQ ID NO: 61)
>LON2_BACSU [Bacillus subtilis]
MSWTGIALFIQLFFGIIIGLYFWNLLKNQRTQKVTIDKESKKEMEQLRKMRAISLSEPLSEKVRPKSFKDIVGQEDGIKAL
KAALCGPNPQHVIVYGPPGVGKTAAARLVLEEAKKHKQSPFKEQAVFVELDATTARFDERGIADPLIGSVHDPIYQGA
GAMGQAGIPQPKQGAVTHAHGGVLFIDEIGELHPIQMNKMLKVLEDRKVFLDSAYYSEENTQIPNHIHDIFQNGLPADF
RLIGATTRMPNEIPPAIRSRCLEVFFRELEKDELKTVAKTAADKIEKNISEEGLDLLTSYTRNGREAVNMIQIAAGMAVT
ENRKDITIEDIEWVIHSSQLTPKHEQKIGVEPQVGIVNGLAVYGPNSGSLLEIEVSVTAAQDKGSINITGIAEEESIGSQS
KSIRRKSMAKGSVENVLTVLRTMGMKPSDYDIHINFPGGIPIDGPSAGIAMAAGIFSAIHKIPIDNTVAMTGEISLNGLVK
PIGGVIPKIKAAKQSGAKKVIIPYENQQAILKQIDGIEHAVKTFQEVLDEILVNPPTEQKPFHIEINKESV (SEQ ID NO: 62)
>PTG3C_BACSU [Bacillus subtilis]
MFKALFGVLQKIGRALMLPVAILPAAGILLAIGNAMQNKDMIQVLHFLSNDNVQLVAGVMESAGQIVFDNLPLLFAVGV
AIGLANGDGVAGIAAIIGYLVMNVSMSAVLLANGTIPSDSVERAKFFTENHPAYVNMLGIPTLATGVFGGIIVGVLAALLF
NRFYTIELPQYLGFFAGKRFVPIVTSISALILGLIMLVIWPPIQHGLNAFSTGLVEANPTLAAFIFGVIERSLIPFGLHHIFYS
PFWYEFFSYKSAAGEIIRGDQRIFMAQIKDGVQLTAGTFMTGKYPFMMFGLPAAALAIYHEAKPQNKKLVAGIMGSAA
LTSFLTGITEPLEFSFLFVAPVLFAIHCLFAGLSFMVMQLLNVKIGMTFSGGLIDYFLFGILPNRTAWWLVIPVGLGLAVIY
YFGFRFAIRKFNLKTPGREDAAEETAAPGKTGEAGDLPYEILQAMGDQENIKHLDACITRLRVTVNDQKKVDKDRLKQ
LGASGVLEVGNNIQAIFGPRSDGLKTQMQDIIAGRKPRPEPKTSAQEEVGQQVEEVIAEPLQNEIGEEVFVSPITGEIH
PITDVPDQVFSGKMMGDGFAILPSEGIVVSPVRGKILNVFPTKHAIGLQSDGGREILIHFGIDTVSLKGEGFTSFVSEGD
RVEPGQKLLEVDLDAVKPNVPSLMTPIVFTNLAEGETVSIKASGSVNREQEDIVKIEK (SEQ ID NO: 63)
Corynebacterium spp. TRANSMEMBRANE PROTEINS
>A4QFD0_CORGB [Corynebacterium spp.]
MDATFWIIGLVLLVILAIIIVLIVGNQRGKSKTVSFEKPEEEKKQLTQQEKSGNYQAQGGFNFAPAKNEEPVLRDDQKLS
TQPPATPVVPPVVPPVVPPAADPVTEPPVDTAEPGAAAEKPATDEFAAQAEPIAPVKPEKAPEEPSGGEHAVDAASA
EPVPEPTSEVPEAISLDVAETLNAAPEETPAELEEEPEAEWPAAEEAPVFEETVEELDEPVGVAGIEAAENAEVQALE
SVEEEATEAAEAASVTAEFAEAAREQTPVPDTAPEPAPAPIDDIAPASGRIGKLRTRLSRSQNVFGKSVLGILSAGDLD
EDAWEDIEAMLIKADLGSKVTARVVDKLRDKIAERGVGSEAEARAMLRETLIDACRPDLDRSIKAMPNEGKPAVVLVV
GVNGTGKTTTTGKLARVLVSMGHKVVLGAADTFRAAAADQLETWGRRVGAETVRGAEGADPASIAFDAVAKGVERQ
ADVVLIDTAGRLHTSTGLMDQLGKVKRVVEKKAVVDEVLLVLDSTVGQNGMQQARIFREVVEITGVVLTKLDGTAKGG
IVFQVQEELGVPVKLVGLGEGADDLAPFEVEGFVDALLG (SEQ ID NO: 64)
>ATPF_CORGL [Corynebacterium spp.]
MANSIYNLAQADALPLESGNSILFPPLYDIVWSLIPFLIILIVFWKLVLPKFQEVLTEREDRIKGGIQRAEAAQAEAKAALE
KYNAQLAEARTEAAEIREQARERGKQIEAELKDKANEESNRIIESGSKQLLAQREQWNELRREMGQNSINLAEHLLG
DQLSDNVKRSGTIDRFLADLDTVAPNGK (SEQ ID NO: 65)
>COX2_CORGL [Corynebacteriurn spp.]
MEQQNKRGLKRKALLGGVLGLGGLAMAGCEVAPPGGVLGDFLRMGWPDGITPEAVAMGNFWSWVWVAAWIIGIIM
WGLFLTAIFAWGAKRAEKRGEGEFPKQLQYNVPLELVLTIVPIIIVMVLFFFTVQTQDKVTALDKNPEVTVDVTAYQWN
WKFGYSEIDGSLAPGGQDYQGSDPERQAAAEASKKDPSGDNPIHGNSKSDVSYLEFNRIETLGTTDEIPVMVLPVNT
PIEFNLASADVAHSFWVPEFLFKRDAYAHPEANKSQRVFQIEEITEEGAFVGRCAEMCGTYHAMMNFELRVVDRDSF
AEYISFRDSNPDATNAQALEHIGQAPYATSTSPFVSDRTATRDGENTQSNA (SEQ ID NO: 66)
Pseudomonas spp. TRANSMEMBRANE PROTEINS
>WP_034025480.1 [Pseudomonas spp.]
MEFIAEYAGFLARTVTVLAAIIWLVAIVALRGRGRRGAGGHLDVQKLNDFYKDLRERVRHSVLDKASLKALRKEESKA
AKQAKKHPEQKSRVYVLDFDGDIKASATEQLRHEVTAVLSMAGKDDEVVLRLESGGGMVHGYGLAASQLARIRQAG
VPLTVCVDKVAASGGYMMACIGDRILSAPFAILGSIGVVAQLPNVHRLLKKHDIDFEVLTAGEYKRTLTVFGENTEKGR
EKFQEDLEVTHELFKNFVAHYRPQLNMDEIATGEVWLGQAALGKLLVDELKTSDEYLAEQARERDVYQVQFVERKSL
QERVGLAASVVIDRVLVTWWGRLNQQKFWQ (SEQ ID NO: 67)
>WP_090310142.1 [Pseudomonas spp.]
MDIGLREWLIVIGIMVIGGILFDGWRRMRGSKGRLKLKLDRNAANLADDGDDDPELLGPSRVINKHQEPSLDEHDLPE
SALRSSRRDEPLAGDLDLELDEPVAVAERLSSVDERSEEPRSKGQDKPREKTVEKVVEKSEKIVDKAGDKAPASAKP
APVRDQPKPQEVLVISVVARDPHGFRGQALLQSILESGLRFGDMDIFHRHESMAGNGEVLFSMANGVKPGTFDLDNI
DIFTTRAVSFFLGLPGPRHPKQAFDLMVAAARKLSHELDGELKDEQRSVMTAQTIEHYRQRIVEFERRALMHKR
(SEQ ID NO: 68)
>FTSK_PSEAE [Pseudomonas spp.]
MRRKNSDLKDSTTASHAAAWRQQLHSRLKEGVLIALGALCLYLWMALLTYDSADPSWSHSSQVDQVQNAAGRLGA
VSADILFMTLGYFAYLFPLLLGIKTWQVFRRRNLPWEWNTWLFSWRLVGLIFLILAGSALAYIHFHASGHMPASASAG
GAIGQSLGRVAVDALNVQGSTLVFFALFLFGLTVFADLSWFKVMDVTGKITLDFFELIQNAFNRWMGARAERKQLVAQ
LREVDERVADNAPSVPDRREQSKAKERLLEREEALAKHMSEREKRPPPKIDPPPSPKAPEPSKRVLKEKQAPLFVD
TAVEGTLPPLSLLDPAEVKQKSYSPESLEAMSRLLEIKLKEFGVEVSVDSVHPGPVITRFEIQPAAGVKVSRISNLAKDL
ARSLAVISVRVVEVIPGKITVGIEIPNEDRQMVRFSEVLSSPEYDEHKSTVPLALGHDIGGRPIITDLAKMPHLLVAGTT
GSGKSVGVNAMLLSILFKSTPSEARLIMIDPKMLELSIYEGIPHLLCPVVTDMKEAANALRWSVAEMERRYRLMAAMG
VRNLAGFNRKVKDAEEAGTPLTDPLFRRESPDDEPPQLSTLPTIVVVVDEFADMMMIVGKKVEELIARIAQKARAAGIH
LILATQRPSVDVITGLIKANIPTRIAFQVSSKIDSRTILDQGGAEQLLGHGDMLYLPPGTGLPIRVHGAFVSDDEVHRVVE
AWKLRGAPDYIEDILAGVDEGGGGGGSFDGGDGSGEGSEDDPLYDEAVRFVTESRRASISAVQRKLKIGYNRAARMI
EAMEMAGVVIPMNINGSREVIAPAPVRD (SEQ ID NO: 69)
Rhodobacter spp. TRANSMEMBRANE PROTEINS
>RCEH_RHOSH [Rhodobacter spp.]
MVGVTAFGNFDLASLAIYSFWIFLAGLIYYLQTENMREGYPLENEDGTPAANQGPFPLPKPKTFILPHGRGTLTVPGPE
SEDRPIALARTAVSEGFPHAPTGDPMKDGVGPASWVARRDLPELDGHGHNKIKPMKAAAGFHVSAGKNPIGLPVRG
CDLEIAGMNDIWVDIPEQMARFLEVELKDGSTRLLPMQMVKVQSNRVHVNALSSDLFAGIPTIKSPTEVTLLEEDKIC
GYVAGGLMYAAPKRKSVVAAMLAEYA (SEQ ID NO: 70)
>UCRI_RHOCA [Rhodobacter spp.]
MSHAEDNAGTRRDFLYHATAATGVVVTGAAVWPLINQMNASADVKAMSSIFVDVSAVEVGTQLTVKWRGKPVFIRR
RDEKDIELARSVPLGALRDTSAENANKPGAEATDENRSLAAFDGTNTGEWLVMLGVCTHLGCVPMGDKSGDFGGW
FCPCHGSHYDSAGRIRKGPAPRNLDIPVAAFVDETTIKLG (SEQ ID NO: 71)
>Q9L906_RHOCA [Rhodobacter spp.]
MMRAVDRPEFDMSASLSHQEWVRLRTLILLRWAAVVGQLAALIAAYGYYDIALNLPMCIGTIGFAVAANIAAIYLYPES
RRLSQAEVTAILLFDTAQLALLLSLIGGLNNPFALLILVPVTIAATALKLRPTLLLGGATIAMITFVAVFNEPLQTRDGAHI
GLPPMIEFGSWVAIVIGVIFLGAYAHRIAQEIHSMSDALFATQMALSREQKLIDLGGVVAAAAHELGTPLATIKLVSTEL
AEELVDDPELHDDAVLIREQAERCRDILRSMGRAGKDDVHLRTAPLLAVLREAAEPHLDRGKMIYFDWPGEGGSER
QPTIYRYPELVHALRNLIQNAVDFAQTTVWVDAEWTDRSIIVRVTDDGRGYSPNVLNRIGDPFISTRSADAKEYEGMG
LGLFIAKILLERTGAKLRFANGSEPYQKNAPVRGSGAVVELRWHIGRLIAPETGPLGENVPITA (SEQ ID NO: 72)
Vibrio spp. TRANSMEMBRANE PROTEINS
>WP_020333352.1 [Vibrio spp.]
MQELRFVLIVVGVLAIAALLFHGLWSSKKEGKAKFGNKPLGKLEVDQGDADSVEQERSFASAPEDDFEIIRKDRKEPD
FGMDNAFDSKFEADPLLGGVPEDKPFKQEPEEIPSFVAQKSHDDDVVVQEPVMQEPAQPVVEETVPSAFEEPKAEM
HVAEQPAAVSESQEEPKPEPEMQVIVLNVHCAGEEPFVGTELFDSMKQNGLIYGEMDIFHRHVDLSGNGKVLFSVAN
MMQPGTLEHGDPAEFTTKGISFFMTLPCFGEAEQNFNLMLRTAQQIADDMGGNVLDDQRNLMTPDRLAAYRRQIVE
FKTANA (SEQ ID NO: 73)
>WP_065296230.1 [Vibrio spp.]
MLIRLIYTLLLALASPILLFGLYKSKPNKPKFGPRWKEHFGITPKLNTDQRPIWIHAVSVGESIAAAPLIKALKKQNPEQPI
LVTTTTSTGAEQVEKLGGLVEHRYMPIDFNFAVKGFLKATNPKQMLIIETELWPNTLNITVHKAGIPISVVNARLSEKSC
NNYAKVQALFNLLHPCLTQVLCQAESDAARFERLGVERNKLSVTGSIKFDIQISDDVKAKGKELRSTLGENRPVWIAA
STHKGEDEQVLEAHKQVLELHPNALLVLVPRHPERFDNVFELCQNQGFETVRRTSKSDVTESTQVYLGDTMGEMLT
LIGSADVCFMGGSLIGYKVGGHNVLEPAALGVPIITGPSYFNFSEIIDALRNSSAIIIAQDYHAIAEHVSNLIPSRRDRQSII
SNAQNIVKTNQGAIVRTLNKVNF (SEQ ID NO: 74)
>MSBA_VIBCH [Vibrio spp.]
MSLHSDESNWQTFKRLWTYIRLYKAGLWSTIALVINAAADTYMISLLKPLLDEGFGNAESNFLRILPFMILGLMFVRGL
SGFASSYCLSWVSGNVVMQMRRRLFNHFMHMPVRFFDQESTGGLLSRITYDSEQVAGATSRALVSIVREGASIIGLL
TLMFWNSWQLSLVLIVVAPVVAFAISFVSKRFRKISRNMQTAMGHVISSAEQMLKGHKVVLSYGGQEVERKRFDKVS
NSMRQQTMKLVSAQSIADPVIQMIASLALFAVLFLASVDSIRAELTPGIFTWFSAMFGLMRPLKALTSVTSEFQRGMA
ACQTLFGLMDLETERDNGKYEAERVNGEVDVKDVTFTYQGKEKPALSHVSFSIPQGKTVALVGRSGSGKSTIANLFT
RFYDVDSGSICLDGHDVRDYKLTNLRRHFALVSQNVHLFNDTIANNIAYAAEGEYTREQIEQAARQAHAMEFIENMPQ
GLDTVIGENGTSLSGGQRQRVAIARALLRDAPVLILDEATSALDTESERAIQAALDELQKNKTVLVIAHRLSTIEQADEIL
VVDEGEIIERGRHADLLAQDGAYAQLHRIQFGE (SEQ ID NO: 75)
Zymomonas spp. TRANSMEMBRANE PROTEINS
>CCME_ZYMMO [Zymomonas spp.]
MQAKHQRLILGIIALAAVIAAGFLALVAFKKQAAYFFTPTDAVKAHLPANRNIRLGGMVERGSLIREKDGVTIHFRVTDG
YQKIAVSYRGIVPDLFREGSGVVADGHFDPSGSFTAETILAKHDERYMPPVTQQQAAATQTTLQEK (SEQ ID NO: 76)
>WP_023593463.1 [Zymomonas spp.]
MTSRSAMRRGFFIAVAGHVLLLLFFLIRFSLQDKRQSQPVDTIEVTFAHGVGIHSSSPEASSEAARATTAPKIGEEKEA
APAPKKETPKEADSQPKAQTQNPAKAEAKVENKPVAKPEPQPKAKPVETPKPAPVAKPEPQPREAAVAPAQKAKVT
AAEKTQPKAESKTPSKPHNDVKNLMESFSKSQVNVSDHKAGSNTESNKGKEKSNASDSGKSKSDNSRNSGSRLGN
DFLKGILGGTGTSSKPQGSSISGPALNGLGAAIKRQVQPCYDLGALGGTDAMRIITVLRLRFNPDGSVNGVPEMIEQT
GLTSDNRSYARQMAEISRRAVLRCAPLHLPAELYKGGWDEIDIGFIPAQMH (SEQ ID NO: 77)
>WP_011240504.1 [Zymomonas spp.]
MFAAIGFTILLVMVFGGFAITGGHLGPVLEAIPHELIVIGGASVGSWAGNSMLELKLLLGGFKSVFKGPKFNKEDYQNT
IFLVGKLMRTLRTEGPVALESHVENPESSAFFAEYPKLLTDKILVSLITDTLRLWVSSGTLDPNAVEDVIDNTIKTHHSE
TSLPADILQNLSEALPAFGIVAAVLGVVKTMGSIDQPPPILGAMIGSALVGTFLGVLLAYGIVAPMANRCRAVIDSDAHIY
QVIKQMIISSMQGHPLPLVIEAARSDIPEENKPEFAEIFDGMRGR (SEQ ID NO: 78)
Pichia pastoris TRANSMEMBRANE PROTEINS
>ANZ73349.1 [Pichia pastoris]
MNSFPASDNSNTPAVSLEKLKETDGSFKGMASGLLEDVMKGNPYFAAGGGLMLLGTGLALLKSGITRVSGLAYRQM
LVDLEIPSKDKSYLWFLEWMSQYKHRSSRHLSVETNYTQHNNGSISTSFSLVPGPGKHLIKYEGAWMLINRERSGKLL
DMINGTPFETITLTTLYRDRNKFPSLLEEAKKMALKTREGKTVIYTSWGQEWRPFGQPRMKRLIDSWLDKGIKESIID
DVQDFLTSGQWYHDRGIPYRRGYLLYGPPGSGKTSFIQSLAGYLDYNICILNLSETNLTDDRLNYLMNHIPERSILLLE
DVDAAFNKRSQTDEKGYSSGVTFSGLLNALDGVASAEEMLTFMTSNHPERLDPALLRPGRVDYKVLIDNASIYQIERM
FLRFYGETHRELCDEFLEQFKTLGLPTVSAAQLQGLFVYNKRDPKKAIEMVEVLKQPNHVF (SEQ ID NO: 79)
>SEC11_KOMPG [Pichia pastoris]
MNIRQQLVQLLNLAMVLSTAFMFWKGLGLVINSNSPIWVLSGSMEPAFQRGDILFLWNRDKYVDIGDVWYEVKGK
PIPIVHRVLREHKVTNKDRKVRQLLLTKGDNNPTDDLSLYAHKSNYLDRDEDVLGTVKAYLPKVGYVTILITENKYAKL
GLLGLMALSTLLTRE (SEQ ID NO: 80)
>SHOLKOMPG [Pichia pastoris]
MSCSFQLSLVPSPPNLKMSIRNIINDPFATGSLSLAAMSWIITFISSIVADVQGSFPKLTWWGIVFELFVILFLAVLYIVDDI
QPHRLSIVGFLAIATVYTTNSANSLVYSNTSAKNCAAAGSILLSMINLIWLFYYGTDIANSVLVARVFGKQGSNPFTTPQ
PNRTYTPNDINNTNTNYMSSGQLTGLEKGVDSNAYGNHDDSDDELSADDHYPITVRALYNYDANPEDINELSLKQGE
VFKVKDTAGKWWQAKKQSGELGICPSNYVETLH (SEQ ID NO: 81)
Saccharomyces cerevisiae TRANSMEMBRANE PROTEINS
>BCSLYEAST [Saccharomyces cerevisiae]
MSDKPIDIQYDKQATPNLSGVITPPTNETGNDSVREKLSKLVGDAMSNNPYFAAGGGLMILGTGLAVARSGIIKASRVL
YRQMIVDLEIQSKDKSYAWFLTWMAKHPQRVSRHLSVRTNYIQHDNGSVSTKFSLVPGPGNHWIRYKGAFILIKRERS
AKMIDIANGSPFETVTLTTLYRDKHLFDDILNEAKDIALKTTEGKTVIYTSFGPEWRKFGQPKAKRMLPSVILDSGIKEGI
LDDVYDFMKNGKWYSDRGIPYRRGYLLYGPPGSGKTSFIQALAGELDYNICILNLSENNLTDDRLNHLMNNMPERSIL
LLEDIDAAFNKRSQTGEQGFHSSVTFSGLLNALDGVTSSEETITFMTTNHPEKLDAAIMRPGRIDYKVFVGNATPYQVE
KMFMKFYPGETDICKKFVNSVKELDITVSTAQLQGLFVMNKDAPHDALKMVSSLRNANHIF (SEQ ID NO: 82)
>YFH6_YEAST [Saccharomyces cerevisiae]
MCLEPISLWFGSLVFFFGLVKYFKRGERQRTRGILQPEYKDKYYYSKEKGEEMGEVANVNEIPVKIRNHKYPAKEHN
LRVKDLLLNRNPKLSKISTAFFIAGEELEGNKYCDTNKDFRQNRYFYHLSGVDIPASAILFNCSTDKLTLFLPNIDEEDVI
WSGMPLSLDEAMRVFDIDEALYISDLGKKFKELQDFAIFTTDLDNVHDENIARSLIPSDPNFFYAMDETRAIKDWYEIES
IRKACQISDKSHLAVMSALPIELNELQIQAEFEYHATRQGGRSLGYDPICCSGPACGTLHYVKNSEDIKGKHSILIDAGA
EWRQYTSDITRCFPTSGKFTAEHREVYETVLDMQNQAMERIKPGAKWDDLHALTHKVLIKHFLSMGIFKKEFSEDEIF
KRRASCAFYPHGLGHMLGLDVHDVGGNPNYDDPDPMFRYLRIRRPLKENMVITNEPGCYFNQFLIKEFLEKHPERLE
VVDMSVLKRYMYVGGVRIEDDILVTKDGYENLIGITSDPDEIEKIVQKGLKKPRSGFHVIV (SEQ ID NO: 83)
>YB85_YEAST [Saccharomyces cerevisiae]
MVSRFYQIPGTHRPSSAISSSNESSSLLSARRISQTYFNYQATPECQKVSSKYDPDNPNKDKLGTYDGVFVPTALNVL
SILMFLRFGFILGQLGIICTIGLLLLSYTINLLTTLSISAISTNGTVRGGGAYYMISRSLGPEFGGSIGLVFFLGQVFNAGM
NAVGIIEPLLYNLGYSAQGEPPAALGELLPRGHWHEFTYATVILFLCFSVAFVGSQTVSRAGNILFLVLAASIFSIPLSALI
RSPFTEGGISYTGPSWQTFHDNLLPHLTKGAAGSLLKGKETFNDLFGVFFPATAGIFAGAGMSSELRKPSKSIPKGTL
WGLLFTFICYAVVVFSMGCSIPRRSLYDEVQIIQTISSVQWVIFMGEMATSLFSIIVGMLGAAYVLEAIAKDNIIPGLEIFA
HSPLYSLIFTWILTQLCLFSDVNKIATFITMTFLMTFVVMNLACFLLGISSAPNFRPSFKYFNRYTTAIGALLSVVAMLIVD
GISASVLFLAMILLFLFIHYFSPPKSWGDVSQSLIYHQVRKYLLRLRQDNIKYWRPQILLFVDNPRTSWNLIRFCNHLKK
GGLYILGHVAVTADFPKQLNELKTQQKAWMKIRDMAAIKAFVQVGTGPSLIWGIRNVFIGSGLGGMKPNITVVGFFDL
ESYRKHIPQSRSQNNLQKQVEIKATVPRSTCSDVKINVPLPTDECKNETKVNVQQWVQIVEDLSLMQSNIAIAHGFKN
LEIPNKRDSCFPKKTIDLYPIQMCGKVEAKGDQPAAITTNFDTYTLILQLAAILVTVPEWKRTHSLRVILFVEQEYHRTNE
TQRMKKLLQVLRIDAEVLVVSLDQFRVYNTIVKGDPIVFDYVNSKLADNEWWKDLVEARDTLKPKRRFSTIEPQTIAKQ
FTQSRKYTSGVQKLGVSFTMNTRMPTNRIDTPCESEDSDLDTDLTSIRDAFSASTNISVGKDLTTKSKTGSDRTNLLV
KNLQSDVSTQSLRPVFSSNTLPRTRVVEDGTGEQPTLIPIAEPDLSNGNGTGSGIGNGNKLKKPVLPELSPCCSKDSL
VTAMQNLGFNDLPSTAQHLVLNDVMTQMSKSSDLIFSTLPVPALGTHEDHDASLQYVEDLDIWLEGLPPCMLINSQT
MTVTTAL (SEQ ID NO: 84)
Yarrowia lipolytica TRANSMEMBRANE PROTEINS
>XP_502343.1 [Yarrowia lipolytica]
MVNFGAQSIRQTLVQLLGFAAIFTSSYMFYKGLSIVANSESPLVVVLSGSMEPAYQRGDVLLLWNRQKHVDVGEVVV
YNIDGRTTPIVHRVLRSHASDNKQLLLTKGDNNAVDDVSFYGGRNQYLDREKEVVGWKGYLPLVGYITILLAENQYF
KYGLLGITGLLAFIQGE (SEQ ID NO: 85)
>XP_502366.1 [Yarrowia lipolytica]
MADATTAAATAGSSAASAPAAAPSGNFLTSMLGENSGFLENPLFTGGMGLMVLGAVVALGRTGIVQGSQFLQRQLL
VDLEIASKDRSYAWFLEWMSHHPQRSSRHLAVQTTVKQHASGSFTTQFSLVPGPGRHLIRYKGAFMLVKRERSNRL
LDMNSGSPFETITLTTLYRDRYVFQELLAEAQQRAQKMQAGKTVIYTSFGPEWRPFGQPRRKRELDSVILDKGVSESI
VEDVNDFLKNSQWYHDRGIPYRRGYLLYGPPGSGKSSFIQALAGELDYNICILNLAEATLTDDRLNHLMNHVPERTFL
LLEDIDSAFNERKQSADQGYHSGVTFSGLLNALDGVASAEERIIFMTTNHPERLDPALIRPGRVDFKECIDNATEYQAE
KMFMRFFPGEEKLCNEFIQTLKANNKLVSTAQMQGLFVMNKTDPVGAIHMAQYLPENEGTPSPASEPPVHKGV (SEQ
ID NO: 86)
>XP_500492.1 [Yarrowia lipolytica]
MPSLTWDIQKPVREETAPIRGGPSNTPDHNYGALQRPCTVQQEEDRLIGDFYKRHNSVAGDKPADTGDEKLGTFSG
VFMPTTLNVLSILMFLRFGFILGQVGILGMFALLVLSYAIDLLTTLSISAIATNGTVRGGGAYYMISRSLGPEFGGAIGVVF
FFGQVLNAGLNVAGFCQPILSSFGQNAGGFFPEGYWYEFFYATGVLLFCTSICMFGSGLFSQAGKVLFVILIVATVSVP
LSVFFVKPFLVTKLDIWYMGPSWDVFSDNLLPRFTTGAVGSDLPPGQMETFTSLFGVFFPATAGIFAGASMSGDLKR
PSYSIPKGTLSGLGLTFILYAATILGMGVAIPRALLYKDISVIETVNLSKWLILFGEMSTSLFSSMVGVIGAAYVLQAIAKD
ALVPYTSFLASEINGLPIPAVFTTYILTQLTLFFPLNRLATFITMAYLMTFWINLACFLLKIASAPNFRPSFKYFSSTTAFL
GAVSCIASMFIADGWASIGAIVILAFLFILIHYVSPPKPWGDVSQALLYHQVRKYLLRLRQDHVKFWRPQILLLVDDPRS
AWGLIKFCNYLKKGGLYILGHVVITKDFQETFKEVKKQQQSWTKLRDMTGAKAFVQIACSPDWWGARNVFLGSGLG
GMKPNITILGSLRDKSQPLDLHRAQTIDMEALPTDNCRKESNIRVTQWVNIIEDIIAMQGNVAIARGFMGMELPGKNIKT
TQKYIDLYPIQMSAQWDENGTSTMTINFDTYTLILQMGAILRTVPLWKERYTLRVIVFVEFEDAVEEERERVSTLLDTL
RIKAKILVLCLNSGNYAAYECIIKGTSNPAVESKLSEQDWWSELVEARESNKPYAFTRKMEEHKVIAIDHKRRHTFSSL
SHLGASFSLRANSISSAGTFDSEGFSDYDENSSVIDDEEDEDEDDTSELEQNTSALHRHNSLFSTSRPLDRVKDKSR
DDESSSGRSSPSSSTHHERHNSNKPAFTSQAIPKTKVNDNDDDEGNITVMFEHGDDPAHPNVLSFNDVPARAQHIILN
DMMATLSSKEDTWIFSTLPAPSMGTHRSERESLDFVDSLELWCQDLPPVLLLHCQTMTVITAL (SEQ ID NO: 87)

1. A method for biosynthesis of one or more chemical species in a bacteria or yeast host cell comprising:
expressing one or more biosynthetic pathways in the bacteria or yeast host cell, the one or more biosynthetic pathways comprising at least one membrane-anchored P450 enzyme having a transmembrane domain derived from a bacteria or yeast inner membrane cytoplasmic C-terminus protein, and
culturing the bacteria or yeast host cell to produce the one or more chemical species from the biosynthetic pathway(s).

2. The method of claim 1, wherein the bacteria or yeast host cell does not exhibit a substantially stressed phenotype during the culturing.

3. The method of claim 1, wherein the bacteria or yeast host cell expresses at least two, at least three, or at least four recombinant enzymes.

4. The method of claim 3, wherein the biosynthetic pathway(s) produce a secondary metabolite through the overexpression of at least two foreign genes.

5. (canceled)

6. The method of claim 1, wherein the bacteria or yeast host cell contains an overexpression of at least two bacteria or yeast genes.

7. The method of claim 6, wherein the bacteria overexpresses at least one gene in the MEP pathway.

8. The method of claim 6, wherein at least one gene is expressed by a strong promoter.

9.-10. (canceled)

11. The method of claim 1, wherein at least one P450 enzyme is not strongly expressed.

12. The method of claim 1, wherein the bacteria or yeast host cell expresses at least two P450 enzymes, which are optionally derived from plant P450 enzymes.

13. The method of claim 12, wherein the bacteria or yeast host cell expresses a membrane-anchored P450 selected from CiVO, HmPO, LsGAO, BsGAO, NtEAO, SrKO, SrKAH, AtKAH, ZzHO, CpVO, MsL6OH, NtVO, StVO, AtKO, Ci2VO, AaAO, and Taxus 5-alpha hydroxylase, or derivative thereof.

14. The method of claim 1, wherein the biosynthetic pathway produces a secondary metabolite selected from a terpenoid, alkaloid, cannabinoid, steroid, saponin, glycoside, stilbenoid, polyphenol, antibiotic, polyketide, fatty acid, or non-ribosomal peptide.

15. The method of claim 14, wherein the biosynthetic pathway produces a terpenoid selected from a monoterpenoid, a sesquiterpenoid, diterpenoid, a sesterpenoid, or a triterpenoid.

16.-22. (canceled)

23. The method of claim 1, wherein IbpA is not overexpressed during the culturing.

24. The method of claim 1, wherein the culturing is conducted at 30° C. or greater.

25.-32. (canceled)

33. The method of claim 1, wherein the cell expresses a single CPR protein.

34. (canceled)

35. The method of claim 1, wherein at least one membrane anchor is a single pass transmembrane domain derived from a bacteria gene selected from:
a B. subtilis gene selected from ATPL_BACSU and LON2_BACSU, or a derivative thereof;
a Corynebacterium spp. gene selected from A4QFD0_CORGB and ATPF_CORGL, or a derivative thereof;
a Pseudomonas spp. gene selected from WP_034025480.1 and WP_090310142.1, or a derivative thereof;
a Rhodobacter spp. gene selected from RCEH_RHOSH or UCRI_RHOCA, or a derivative thereof;
a Vibrio spp. gene selected from WP_020333352.1 or WP_065296230.1, or a derivative thereof; and
a Zymomonas spp. gene selected from CCME_ZYMMO or WP_023593463.1, or a derivative thereof.

36. The method of claim 1, wherein at least one membrane anchor is a multi-pass transmembrane domain derived a bacteria gene selected from:
the B. subtilis gene PTG3C_BACSU, or a derivative thereof;
the Corynebacterium spp. gene COX2_CORGL, or a derivative thereof;
the Pseudomonas spp. gene FTSK_PSEAE, or a derivative thereof;
the Rhodobacter spp. gene Q9L906_RHOCA, or a derivative thereof;
the Vibrio spp. gene MSBA_VIBCH, or a derivative thereof; and
the Zymomonas spp. gene WP_011240504.1, or a derivative thereof.

37. The method of claim 1, wherein at least one membrane anchor is a single pass transmembrane domain derived from a yeast gene selected from:
a P. pastoris gene selected from ANZ73349.1 and SEC11_KOMPG, or a derivative thereof;
a S. cerevisiae gene selected from BCS1_YEAST and YFH6_YEAST, or a derivative thereof; and
a Y. lipolytica gene selected from XP_502343.1 and XP_502366.1, or a derivative thereof.

38. The method of claim 1, wherein at least one membrane anchor is a multi-pass transmembrane domain derived a yeast gene selected from:
the P. pastoris gene SHO1_KOMPG, or a derivative thereof;
the S. cerevisiae gene YB85_YEAST, or a derivative thereof; and
the Y. lipolytica gene XP_500492.1, or a derivative thereof.

39. The method of claim 1, wherein the P450 enzyme has a deletion of part or all of its native N-terminal transmembrane domain.

40.-51. (canceled)

52. A method for producing a product comprising one or more terpenoid compounds, comprising:
expressing a terpenoid biosynthetic pathway in a bacteria or yeast host cell, wherein the biosynthetic pathway comprising at least one membrane-anchored P450 enzyme having a transmembrane domain derived from a bacteria or yeast inner membrane cytoplasmic C-terminus protein; and
culturing the bacteria or yeast host cell to produce the one or more terpenoids from the biosynthetic pathway;
recovering the terpenoid(s) from the culture; and
incorporating the terpenoid into a product.

53.-99. (canceled)

100. A bacterial or yeast host cell expressing one or more recombinant biosynthetic pathways, where the biosynthetic pathways comprise at least one membrane-anchored P450 protein having a transmembrane domain derived from a bacteria or yeast inner membrane cytoplasmic C-terminus protein.

101.-140. (canceled)

141. A plant P450 enzyme comprising an N-terminal truncation and a transmembrane region derived from a bacteria or yeast inner membrane cytoplasmic C-terminus protein.

142.-153. (canceled)

154. A polynucleotide encoding the enzyme of claim 141.
